(19) United States
(12) Patent Application Publication
(10) Pub. No.: US 2023/0212142 A9
(43) Pub. Date: Jul. 6, 2023

(54) Substituted 5-Cyclopropyl-1H-pyrazole-3-yl-amine Derivatives as Selective CDK12/13 Inhibitors

(71) Applicant: Aurigene Discovery Technologies Limited, Bangalore (IN)

(72) Inventors: Ramulu Poddutoori, Jammikunta (IN); Susanta Samajdar, Bangalore (IN); Subhendu Mukherjee, Hooghly (IN)

(73) Assignee: Aurigene Discovery Technologies Limited, Bangalore (IN)

(21) Appl. No.: 17/600,024

(22) PCT Filed: Mar. 31, 2020
(86) PCT No.: PCT/IB2020/053038

§ 371 Date: Sep. 29, 2021

(30) Foreign Application Priority Data
Apr. 1, 2019 (IN) 201941013150

(51) Int. Cl. C07D 401/12; C07D 401/14; C07D 403/12; C07D 231/40

(52) U.S. Cl. CPC C07D 401/12; C07D 401/14; C07D 403/12; C07D 231/40

(57) ABSTRACT

The present invention provides 5-cyclopropyl-1H-pyrazol-3-yl-amine derivatives of formula (I), which are therapeutically useful as selective CDK12/13 inhibitors. These compounds are useful in the treatment and/or prevention of diseases and/or disorders associated with CDK12/13 in a mammal. The present invention also provides the preparation of the compounds and pharmaceutical compositions that have at least one of the 5-cyclopropyl-1H-pyrazol-3-yl-amine derivatives of formula (I) or a pharmaceutically acceptable salt, an N-oxide or a stereoisomer thereof.

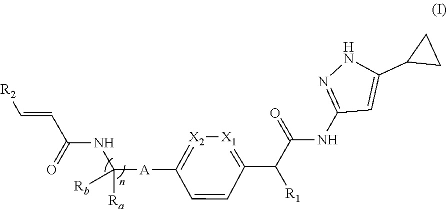

This application claims the benefit of Indian provisional application number 201941013150, filed on 1 Apr. 2019; the specifications of which are hereby incorporated by reference in their entirety and for all purposes.

### FIELD OF THE INVENTION

This invention relates to substituted 5-cyclopropyl-1H-pyrazol-3-yl-amine derivatives useful for treatment of cancer and inflammatory diseases associated with CDK12/13. The invention also provides pharmaceutically acceptable compositions comprising compounds of the present invention and methods of using said compositions in the treatment of diseases associated with CDK12/13.

### BACKGROUND OF THE INVENTION

Cyclin dependent kinases (CDKs) are a family of Ser/Thr kinases that integrate various signal transduction pathways and play a key role in several key cellular processes. CDK12 and its orthologue CDK13 belong to the class of ‘transcriptional’ CDKs. CDK12/Cyclin K regulates transcriptional elongation, pre-mRNA splicing and alternate splicing. The Cancer Genome Atlas (TCGA) project has identified CDK12 mutations in several breast and ovarian cancers, implicating its role as tumour suppressor. Mutation of CDK12 in serous ovarian carcinoma is associated with decreased expression of DNA damage response (DDR) genes such as BRCA1, FANCI, ATM, ATR or FANCD2 and increased sensitivity to PARP inhibitors. (Cancer Res, 2016, 76(7) 1182; Nucleic Acids Research, 2015, Vol. 43, 2575-2589). Hence, maintenance of genomic stability appears to be the key role of this protein.

Transcription of protein-coding genes is controlled by RNA Polymerase II. Phosphorylation of residues in its C-terminal domain (CTD) orchestrate the production of mature mRNA transcript. Phosphorylation of Ser2, which promotes elongation of RNA Pol II through the gene body, is a key mechanism of CDK12 transcriptional regulation (Genes & Development 2010, 24:2303-2316). As a consequence, CDK12 knockdown has also been associated with downregulation of genes involved in homologous recombination (Genes &Development 2011, 25:2158-2172). The emergence of increasingly significant role of CDK12 in genomic stability and oncogenesis provides new insight towards deciphering the function of CDK12 in genome maintenance and oncogenesis.

The frequency and distribution of CDK12 protein expression was assessed by Immuno Histo Chemistry (IHC) in independent cohorts of breast cancer and this was correlated with outcome and genomic status. It was found that 21% of primary unselected breast cancers were CDK12 high, and 10.5% were absent. CDK12 overexpression in breast cancer cells has been demonstrated to regulate splicing of pre-mRNA involved in DDR and tumorigenesis. (Nucleic Acids Res., 2017, Jun 20; 45(11):6698-6716). Disruption of Cyclin-Dependent Kinase 12 (CDK12) is known to lead to defects in DNA repair and sensitivity to platinum salts and PARP1/2 inhibitors. Interestingly, absence of CDK12 protein was associated with reduced expression of a number of DDR proteins including ATR, Ku70 /Ku80, PARP1, DNA-PK, and γH2AX, suggesting a novel mechanism of CDK12-associated DDR dysregulation in breast cancer. This may have important therapeutic implications, particularly for triple-negative breast cancers. (Molecular Cancer Therapeutics (2018), 17(1), 306-315).

As transcription is a highly critical cellular process and is controlled by different transcription regulating kinases, it is desirable to have as selective compound as possible to overcome unwanted side effects. For example, CDK-7 is reported to control transcription initiation by phosphorylation of Ser5 and Ser7 residue of RNA polymerase II, whereas CDK-12 is reported to be responsible for elongation of transcription through phosphorylation of Ser2 residue of RNA polymerase II (Nucleic Acids Research, 2015, Vol. 43, No. 5, 2575-2589).

It is reported that inhibition of both initiation and elongation at the same time modulate a much larger number of gene transcriptions. (Popova, T et. al. Cancer Res. 2016, 76, 1882). Consistent with this notion, the findings from a recent study in which genome-scale CRISPR-Cas9 screening across 341 cancer cell lines representing diverse cancer cell types indicated that CDK7 disruption was pan-lethal similar to the depletion seen for known essential genes in the screen, raising some concerns about the therapeutic window of a potent CDK7 inhibitor (Cancer Cell 2018, Vol. 33, 1-15). In contrast to the dependency of 100% of cancer cell lines for CDK7, CDK12 and CDK13 showed differential dependencies only in a subset of cell lines (10.2% and 3.8% respectively) included in the screen supporting the advantages of a selective CDK12/13 inhibitor in a subset of cancer indications over a CDK7 inhibitor (Cancer Cell 2018, Vol. 33, 1-15).

WO2016/193939 discloses compounds that inhibit the activity of certain transcriptional cyclin dependent kinases (CDKs) including CDK7, CDK9, CDK12, CDK13 and CDK18, with a particular focus on the inhibition of transcriptional cyclin dependent kinase-7 (CDK7). The present inventors have found that the compounds disclosed in WO 2016/193939 do indeed inhibit CDK7 and CDK12/13; however, they are not selective towards CDK12/13.

There remains a need in the art to find compounds which selectively inhibit CDK12/13 over other CDKs. It is, therefore, an objective of this invention to provide compounds useful in the treatment and/or prevention or amelioration of diseases and/or disorders associated with CDK12/13.

### SUMMARY OF THE INVENTION

Provided herein are substituted 5-cyclopropyl-1H-pyrazol-3-yl-amine derivatives and pharmaceutical compositions thereof, which are useful as selective CDK12/13 inhibitors for the treatment of diseases associated with CDK12/13.

In one aspect of the present invention, it comprises compounds of formula (I):

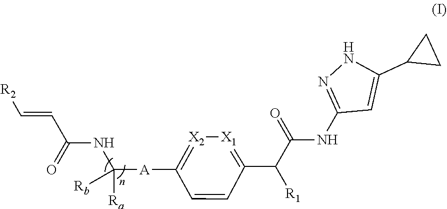

or a pharmaceutically acceptable salt thereof, an N-oxide or a stereoisomer thereof;

wherein,

each of Xᵢ and X₂ is independently CH or N;

A is aryl, heteroaryl or a bond, wherein the aryl and heteroaryl are each optionally substituted with one or more substituents independently selected from halogen, alkyl and alkoxy;

R₁ is hydrogen or alkyl;

R₂ is hydrogen or —(CH₂)ₘ—NR_{c}R_{d};

Rₐ and R_{b} are each independently hydrogen or alkyl; alternatively, Rₐ and R_{b} together with the carbon atom to which they are attached form an optionally substituted cycloalkyl ring;

R_{c} and R_{d} are each independently hydrogen or alkyl; alternatively, R_{c} and R_{d} together with the nitrogen atom to which they are attached form an optionally substituted heterocyclic ring containing 0-2 additional heteroatoms independently selected from N, O and S;

m is 1, 2 or 3; and

n is0, 1 or2.

In yet another aspect, the present invention provides a pharmaceutical composition comprising a compound of formula (I) or a pharmaceutically acceptable salt thereof and at least one pharmaceutically acceptable excipient (such as a pharmaceutically acceptable carrier or diluent).

In yet another aspect, the present invention relates to the preparation of compounds of formula (I).

In yet another aspect of the present invention, provided herein are substituted 5-cyclopropyl-1H-pyrazol-3-yl-amine derivatives of formula (I), which are capable of selectively inhibiting CDK12/13 and therapeutic use thereof.

In a still further aspect, the invention provides methods of treating diseases and/or disorders or conditions mediated by CDK12/13 in a subject comprising administration of compounds of formula (I) or pharmaceutical compositions thereof.

### DETAILED DESCRIPTION OF THE INVENTION

The present invention provides substituted 5 -cyclopropyl-1H-pyrazol-3 -yl-amine derivatives of formula (I), which are useful as selective CDK12/13 inhibitors.

The present invention further provides pharmaceutical compositions comprising the said substituted 5-cyclopropyl-1H-pyrazol-3-yl-amine compounds of formula (I) and their derivatives as therapeutic agents.

In first embodiment, the present invention provides compounds of formula (I),

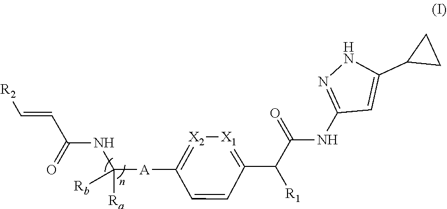

or a pharmaceutically acceptable salt thereof, an N-oxide or a stereoisomer thereof; wherein,

each of X₁ and X₂ is independently CH or N;

A is aryl, heteroaryl or a bond, wherein the aryl and heteroaryl are each optionally substituted with one or more substituents independently selected from halogen, alkyl and alkoxy;

R₁ is hydrogen or alkyl;

R₂ is hydrogen or —(CH₂)ₘ—NR_{c}R_{d};

Rₐ and R_{b} are each independently hydrogen or alkyl; alternatively, Rₐ and R_{b} together with the carbon atom to which they are attached form an optionally substituted cycloalkyl ring;

R_{c} and R_{d} are each independently hydrogen or alkyl; alternatively, R_{c} and R_{d} together with the nitrogen atom to which they are attached form an optionally substituted heterocyclic ring containing 0-2 additional heteroatoms independently selected from N, O and S;

m is 1, 2 or 3; and

n is 0, 1 or 2.

In another embodiment of the present invention, it provides compounds of formula (I),

wherein,

each of X₁ and X₂ is independently CH or N;

A is aryl, heteroaryl or a bond; wherein, each aryl and heteroaryl is optionally substituted with one or more substituents independently selected from halogen, alkyl and alkoxy;

R₁ alkyl;

R₂ is hydrogen or —(CH₂)ₘ—NR_{c}R_{d};

Rₐ and R_{b} are each independently hydrogen or alkyl; alternatively, Rₐ and R_{b} together with the carbon atom to which they are attached form an optionally substituted cycloalkyl ring;

R_{c} and R_{d} are each independently hydrogen or alkyl; alternatively, R_{c} and R_{d} together with the nitrogen atom to which they are attached form an optionally substituted heterocyclic ring containing 0-2 additional heteroatoms independently selected from N, O and S;

m is 1, 2 or 3; and

n is 0, 1 or 2.

In another embodiment of the present invention, it provides compounds of formula (I),

wherein,

each of X₁ and X₂ is independently CH or N;

A is C₆-C₈ aryl or 5 to 8 membered heteroaryl; wherein, each aryl and heteroaryl is optionally independently substituted with one or more halogen;

R₁ is hydrogen or alkyl;

R₂ is hydrogen or —(CH₂)ₘ—NR_{c}R_{d};

Rₐ and R_{b} are each independently hydrogen or alkyl; alternatively, Rₐ and R_{b} together with the carbon atom to which they are attached form an optionally substituted cycloalkyl ring;

R_{c} and R_{d} are each independently hydrogen or alkyl; alternatively, R_{c} and R_{d} together with the nitrogen atom to which they are attached form an optionally substituted heterocyclic ring containing 0-2 additional heteroatoms independently selected from N, O and S;

m is 1; and

n is 1.

In another embodiment of the present invention, it provides compounds of formula (I),

wherein,

each of X₁ and X₂ is CH;

A is aryl, heteroaryl or a bond; wherein, each aryl and heteroaryl is optionally substituted with one or more substituents independently selected from halogen, alkyl and alkoxy;

R₁ is hydrogen or alkyl;

R₂ is hydrogen or —(CH₂)ₘ—NR_{c}R_{d};

R_{c} and R_{d} are each independently hydrogen or alkyl; alternatively, R_{c} and R_{d} together with the nitrogen atom to which they are attached form an optionally substituted heterocyclic ring containing 0-2 additional heteroatoms independently selected from N, O and S; and m is 1, 2 or 3.

In another embodiment of the present invention, it provides compounds of formula (I), wherein,

A is optionally substituted monocyclic C₆-C₈ aryl or optionally substituted monocyclic 5-8 membered heteroaryl ring.

According to yet another embodiment, specifically provided are compounds of formula (I), wherein,

R₁ is hydrogen or alkyl;

R₂ is hydrogen or —(CH₂)ₘ—NR_{c}R_{d};

R_{c} and R_{d} together with the nitrogen atom to which they are attached form an optionally substituted heterocyclic ring containing 0-2 additional heteroatoms independently selected from N, O and S; and m is 1 to 3.

According to yet another embodiment, specifically provided are compounds of formula (I), wherein,

R₁ is hydrogen or alkyl;

R₂ is hydrogen or —(CH₂)—NR_{c}R_{d};

R_{c} and R_{d} together with the nitrogen atom to which they are attached form an optionally substituted heterocyclic ring containing 0-2 additional heteroatoms independently selected from N, O and S.

In yet another embodiment of the present invention, it provides compounds of formula (I), wherein, A is

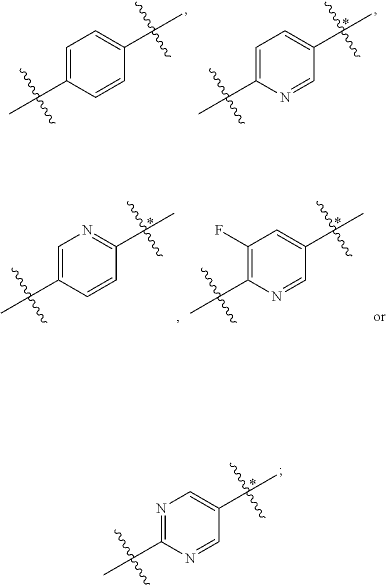 [SMILES: *c1ccc(*)cc1 ; *c1ccc(*)nc1 ; *c1ccc(*)nc1 ; *c1cnc(*)c(F)c1 ; *c1cnc(*)nc1]

wherein * is the point of attachment with ring

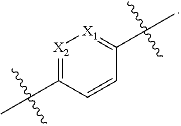

In yet another embodiment of the present invention, it provides compounds of formula (IA),

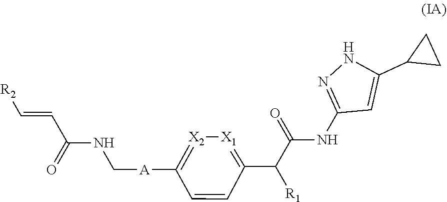

or a pharmaceutically acceptable salt thereof, an N-oxide or a stereoisomer thereof;

wherein, X₁, X₂, A, R₁ and R₂ are same as defined in formula (I).

In yet another embodiment of the present invention, it provides compounds of formula (IB),

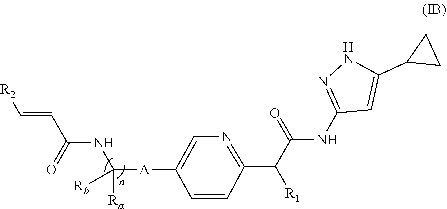

or a pharmaceutically acceptable salt thereof, an N-oxide or a stereoisomer thereof;

wherein, A, R₁, R₂, Rₐ, R_{b} and n are same as defined in formula (I).

In yet another embodiment of the present invention, it provides compounds of formula (IC),

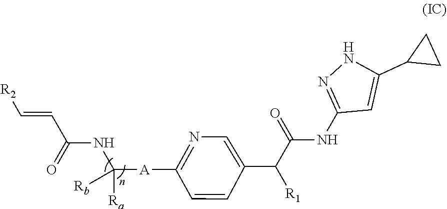

or a pharmaceutically acceptable salt thereof, an N-oxide or a stereoisomer thereof;

wherein, A, R₁, R₂, Rₐ, R_{b} and n are same as defined in formula (I).

In yet another embodiment of the present invention, it provides compounds of formula (ID),

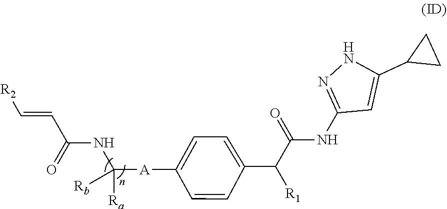

or a pharmaceutically acceptable salt thereof, an N-oxide or a stereoisomer thereof;

wherein, A, R₁, R₂, Rₐ, R_{b} and n are same as defined in formula (I).

In yet another embodiment of the present invention, it provides compounds of formula (IE),

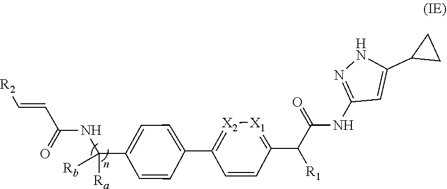

or a pharmaceutically acceptable salt thereof, an N-oxide or a stereoisomer thereof;

wherein, X₁, X₂, R₁, R₂, Rₐ, R_{b} and n are same as defined in formula (I).

In yet another embodiment of the present invention, it provides compounds of formula (IF),

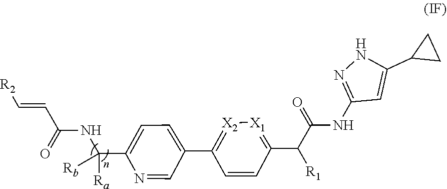

or a pharmaceutically acceptable salt thereof, an N-oxide or a stereoisomer thereof;

wherein, X₁, X₂, R₁, R₂, Rₐ, R_{b} and n are same as defined in formula (I).

In yet another embodiment of the present invention, it provides compounds of formula (IG),

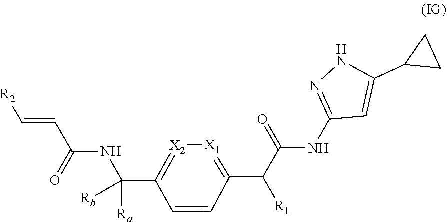

or a pharmaceutically acceptable salt thereof, an N-oxide or a stereoisomer thereof;

wherein, X₁, X₂, R₁, R₂, Rₐ and R_{b} are same as defined in formula (I).

In yet another embodiment of the present invention, it provides compounds of formula (IH),

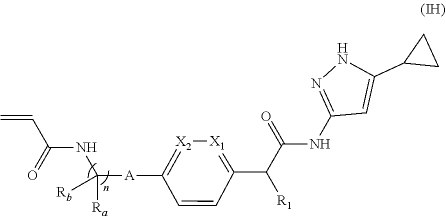

or a pharmaceutically acceptable salt thereof, an N-oxide or a stereoisomer thereof;

wherein, A, R₁, X₁, X₂, Rₐ, R_{b} and n are same as defined in formula (I).

In yet another embodiment of the present invention, it provides compounds of formula (IJ),

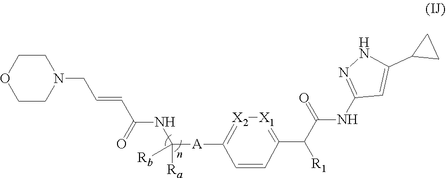

or a pharmaceutically acceptable salt thereof, an N-oxide or a stereoisomer thereof;

wherein, A, R₁, X₁, X₂, Rₐ, R_{b} and n are same as defined in formula (I).

In yet another embodiment of the present invention, it provides compounds of formula (IK),

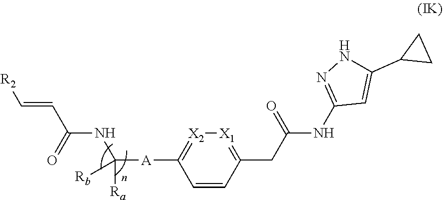

or a pharmaceutically acceptable salt thereof, an N-oxide or a stereoisomer thereof;

wherein, A, R₂, X₁, X₂, Rₐ, R_{b} and n are same as defined in formula (I).

The embodiments below are illustrative of the present invention and are not intended to limit the claims to the specific embodiments exemplified.

In certain embodiments, specifically provided are compounds of formula (I), wherein ring

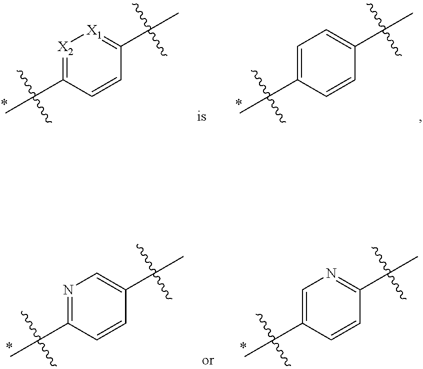

wherein * is the point of attachment with A.

In certain embodiments, specifically provided are compounds of formula (I), wherein A is

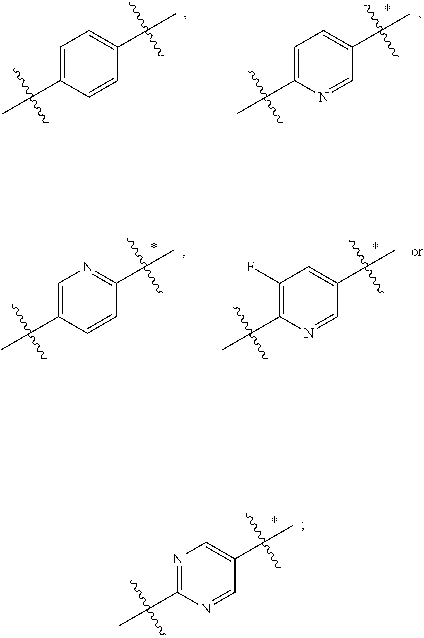 [SMILES: *c1ccc(*)cc1 ; *c1ccc(*)nc1 ; *c1ccc(*)nc1 ; *c1cnc(*)c(F)c1 ; *c1cnc(*)nc1]

wherein * is the point of attachment with ring

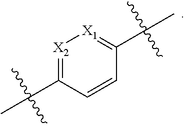

In certain embodiments, specifically provided are compounds of formula (I), wherein A is optionally substituted aryl.

According to the preceding embodiment, specifically provided are compounds of formula (I), wherein the said aryl is phenyl.

According to another embodiment, specifically provided are compounds of formula (I), wherein the ring

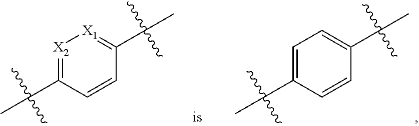

According to yet another embodiment, specifically provided are compounds of formula (I), wherein, the ring

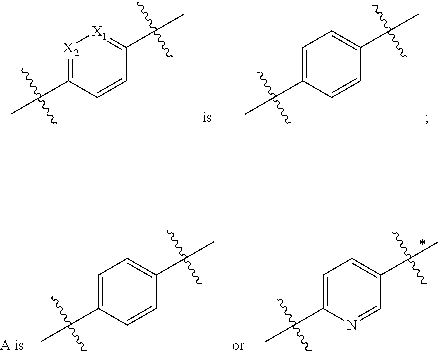

wherein * is the point of attachment with ring

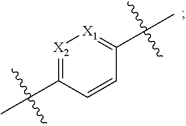

R₁ is alkyl;

R₂ is hydrogen;

Rₐ and R_{b} are each hydrogen; and n is 1.

According to yet another embodiment, specifically provided are compounds of formula (I), wherein the ring

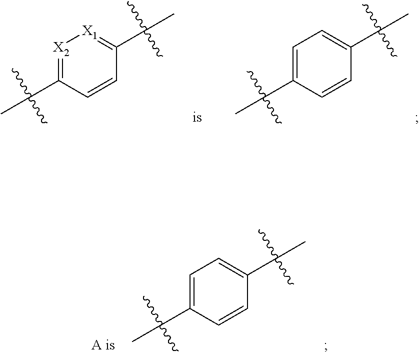

R₁ is alkyl;

R₂ is hydrogen;

Rₐ and R_{b} are hydrogen; and n is 0 to 1.

According to yet another embodiment, specifically provided are compounds of formula (I), wherein the ring

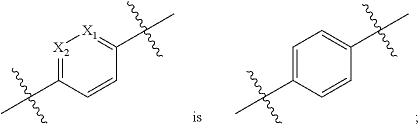

### A is

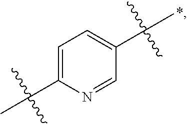 [SMILES: *c1ccc(*)nc1]

wherein * is the point of attachment with ring

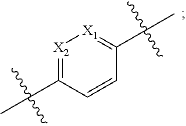

R₁ is alkyl;

R₂ is hydrogen;

Rₐ and R_{b} are hydrogen; and n is 1.

According to yet another embodiment, specifically provided are compounds of formula (I), wherein Rₐ and R_{b} are each independently hydrogen or alkyl; wherein the said alkyl is methyl or ethyl.

According to yet another embodiment, specifically provided are compounds of formula (I), wherein A is phenyl, the ring

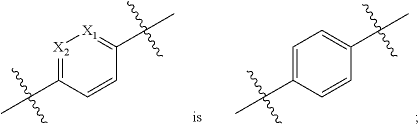

Rₐ and R_{b} are each hydrogen and n is 0 or 1.

According to yet another embodiment, specifically provided are compounds of formula (I), wherein A is optionally substituted heteroaryl.

According to the preceding embodiment, specifically provided are compounds of formula (I), wherein the said optionally substituted heteroaryl is

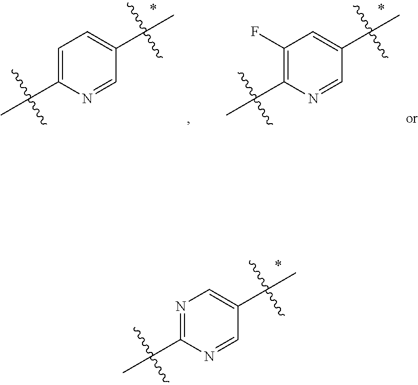 [SMILES: *c1ccc(*)nc1 ; *c1cnc(*)c(F)c1 ; *c1cnc(*)nc1]

wherein * is the point of attachment with ring

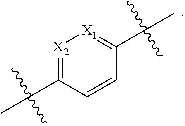

According to yet another embodiment, specifically provided are compounds of formula (I), wherein A is

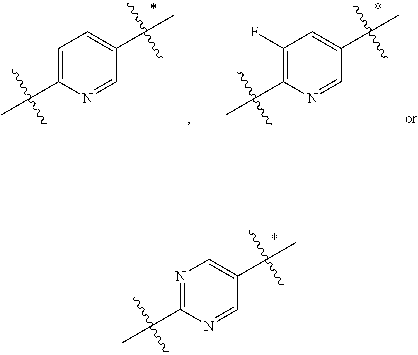 [SMILES: *c1ccc(*)nc1 ; *c1cnc(*)c(F)c1 ; *c1cnc(*)nc1]

wherein * is the point of attachment with ring

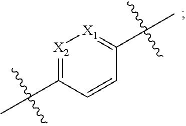

R₁ is alkyl;

R₂ is hydrogen or —(CH₂)ₘ—NR_{c}R_{d};

R_{c} and R_{d} are each independently hydrogen or alkyl; wherein the alkyl is methyl; alternatively, R_{c} and R_{d} together with the nitrogen atom to which they are attached form an optionally substituted heterocyclic ring containing 0-2 additional heteroatoms independently selected from N, O and S; and m is 1, 2 or 3.

According to yet another embodiment, specifically provided are compounds of formula (I), wherein A is

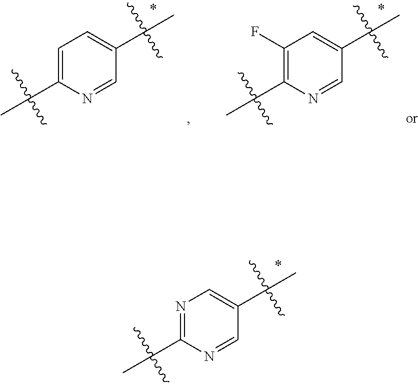 [SMILES: *c1ccc(*)nc1 ; *c1cnc(*)c(F)c1 ; *c1cnc(*)nc1]

wherein * is the point of attachment with ring

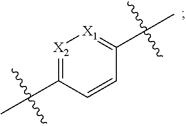

R₁ is alkyl;

R₂ is hydrogen or —(CH₂)—NR_{c}R_{d};

R_{c} and R_{d} together with the nitrogen atom to which they are attached form an optionally substituted ring containing 0-2 additional heteroatoms independently selected from N, O and S.

According to yet another embodiment, specifically provided are compounds of formula (I), wherein,

R₁ is hydrogen or alkyl;

R₂ is hydrogen or —(CH₂)ₘ—NR_{c}R_{d};

R_{c} and R_{d} together with the nitrogen atom to which they are attached form an optionally substituted heterocyclic ring containing 0-2 additional heteroatoms independently selected from N, O and S; and m is 1, 2 or 3.

According to yet another embodiment, specifically provided are compounds of formula (I), wherein A is

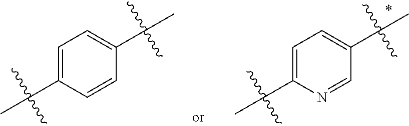 [SMILES: *c1ccc(*)cc1 ; *c1ccc(*)nc1]

wherein * is the point of attachment with ring

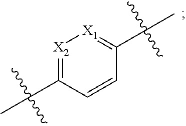

R₁ is alkyl;

R₂ is hydrogen;

Rₐ and R_{b} are each independently hydrogen; and n is 1.

According to yet another embodiment, specifically provided are compounds of formula (I), wherein A is

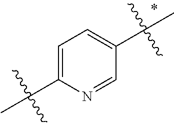 [SMILES: *c1ccc(*)nc1]

wherein * is the point of attachment with ring

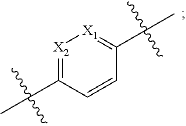

R₁ is alkyl;

R₂ is hydrogen;

Rₐ and R_{b} are each independently hydrogen or alkyl; alternatively, Rₐ and R_{b} together with the carbon atom to which they are attached form an optionally substituted cycloalkyl ring; and

n is 0 to 1.

According to the preceding embodiment, the said alkyl is methyl or ethyl.

According to yet another embodiment, A is a bond.

According to yet another embodiment, when A is a bond, ring

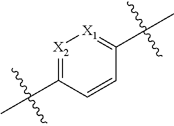

is directly linked to carbon atom containing Rₐ and R_{b}.

According to yet another embodiment, when A is a bond, n is 1.

According to yet another embodiment, when A is a bond, Rₐ and R_{b} are each hydrogen and n is 1.

According to yet another embodiment, specifically provided are compounds of formula (I), wherein R₂ is H or morpholinomethyl.

In yet another embodiment, provided are compounds of formula (I), wherein, the pyrazole ring is in equilibrium stage as shown here:

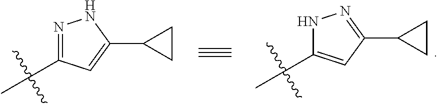

In certain embodiments, a racemic compound is chirally separated to isolate enantiomers.

In certain embodiments, a racemic compound is chirally separated to isolate (R) and (S) isomers.

In certain embodiments, when X₁ and X₂ are each CH and A is absent, then “n” is not “0”.

In certain embodiments, when A is absent, then “n” is 1.

In certain embodiments, when “n” is “0”, then R₂ is not hydrogen.

In one embodiment, the compound of formula (I) is not

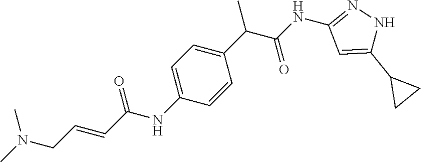 [SMILES: CC(C(=O)Nc1cc(C2CC2)[nH]n1)c1ccc(NC(=O)/C=C/CN(C)C)cc1]

In certain embodiments, the present invention provides a compound of formula (I) selected from:

| Comp | |
|---|---|
| No. | IUPAC NAME |
| 1 | N-(4′-(1-((5-cyclopropyl-1H-pyrazol-3- |
| | yl)amino)-1-oxopropan-2-yl)-[1,1′- |
| | biphenyl]-4-yl)acrylamide; |
| 2 | N-(4′-(1-((5-cyclopropyl-1H-pyrazol-3- |
| | yl)amino)-1-oxopropan-2-yl)-[1,1′- |
| | biphenyl]-4-yl)acrylamide (Isomer-1 |
| | of compound-1); |
| 3 | N-(4′-(1-((5-cyclopropyl-1H-pyrazol-3- |
| | yl)amino)-1-oxopropan-2-yl)-[1,1′- |
| | biphenyl]-4-yl)acrylamide(Isomer-2 |
| | of compound-1); |
| 4 | N-((5-(1-((5-cyclopropyl-1H-pyrazol- |
| | 3-yl)amino)-1-oxopropan-2-yl)[2,3′- |
| | bipyridin]-6′-yl)methyl)acrylamide; |
| 5 | (E)-N-(5-(4-(1-((5-cyclopropyl-1H- |
| | pyrazol-3-yl)amino)-1-oxopropan-2- |
| | yl)phenyl)pyridin-2-yl)-4-morpholinobut- |
| | 2-enamide; |
| 6 | (E)-N-(5-(4-(1-((5-cyclopropyl-1H- |
| | pyrazol-3-yl)amino)-1-oxopropan-2- |
| | yl)phenyl)pyridin-2-yl)-4-morpholinobut-2- |
| | enamide (Isomer-1 of compound-5); |
| 7 | (E)-N-(5-(4-(1-((5-cyclopropyl-1H- |
| | pyrazol-3-yl)amino)-1-oxopropan-2- |
| | yl)phenyl)pyridin-2-yl)-4-morpholinobut- |
| | 2-enamide (Isomer-2 of compound-5); |
| 8 | (S)-N-((5-(4-(1-((5-cyclopropyl-1H- |
| | pyrazol-3-yl)amino)-1-oxopropan-2- |
| | yl)phenyl)pyridin-2-yl)methyl)acrylamide; |
| 9 | N-((4′-(1-((5-cyclopropyl-1H-pyrazol- |
| | 3-yl)amino)-1-oxopropan-2-yl)-[1,1′- |
| | biphenyl]-4-yl)methyl)acrylamide; |
| 10 | N-((4′-(1-((5-cyclopropyl-1H-pyrazol- |
| | 3-yl)amino)-1-oxopropan-2-yl)-[1,1′- |
| | biphenyl]-4-yl)methyl)acrylamide |
| | (Isomer-1 of compound-9); |
| 11 | N-((4′-(1-((5-cyclopropyl-1H-pyrazol- |
| | 3-yl)amino)-1-oxopropan-2-yl)-[1,1′- |
| | biphenyl]-4-yl)methyl)acrylamide |
| | (Isomer-2 of compound-9); |
| 12 | (E)-N-(4′-(1-((5-cyclopropyl-1H-pyrazol- |
| | 3-yl)amino)-1-oxopropan-2-yl)-[1,1′- |
| | biphenyl]-4-yl)-4-morpholinobut-2-enamide; |
| 13A | (E)-N-((5-(4-(1-((5-cyclopropyl-1H- |
| | pyrazol-3-yl)amino)-1-oxopropan-2- |
| | yl)phenyl)pyridin-2-yl)methyl)-4- |
| | morpholinobut-2-enamide; |
| 13 | (E)-N-((5-(4-(1-((5-cyclopropyl-1H- |
| | pyrazol-3-yl)amino)-1-oxopropan-2- |
| | yl)phenyl)pyridin-2-yl)methyl)-4- |
| | morpholinobut-2-enamide (Isomer-1 of |
| | compound-13A); |
| 14 | (E)-N-((5-(4-(1-((5-cyclopropyl-1H- |
| | pyrazol-3-yl)amino)-1-oxopropan-2- |
| | yl)phenyl)pyridin-2-yl)methyl)-4- |
| | morpholinobut-2-enamide (Isomer-2 of |
| | compound-13A); |
| 15 | (E)-N-((4′-(1-((5-cyclopropyl-1H-pyrazol- |
| | 3-yl)amino)-1-oxopropan-2-yl)-[1,1′- |
| | biphenyl]-4-yl)methyl)-4-morpholinobut- |
| | 2-enamide; |
| 16 | (E)-N-((4′-(1-((5-cyclopropyl-1H- |
| | pyrazol-3-yl)amino)-1-oxopropan-2-yl)-[1,1′- |
| | biphenyl]-4-yl)methyl)-4-morpholinobut- |
| | 2-enamide (Isomer-1 of compound- |
| | 15); |
| 17 | (E)-N-((4′-(1-((5-cyclopropyl-1H-pyrazol- |
| | 3-yl)amino)-1-oxopropan-2-yl)-[1,1′- |
| | biphenyl]-4-yl)methyl)-4-morpholinobut- |
| | 2-enamide (Isomer-2 of compound- |
| | 15); |
| 18 | (S)-N-(1-(5-(4-(1-((5-cyclopropyl-1H- |
| | pyrazol-3-yl)amino)-1-oxopropan-2- |
| | yl)phenyl)pyridin-2-yl)cyclopropyl)acrylamide; |
| 19 | N-(1-(5-(4-((S)-1-((5-cyclopropyl-1H- |
| | pyrazol-3-yl)amino)-1-oxopropan-2- |
| | yl)phenyl)pyridin-2-yl)propyl)acrylamide; |
| 20 | N-(1-(5-(4-((S)-1-((5-cyclopropyl-1H- |
| | pyrazol-3-yl)amino)-1-oxopropan-2- |
| | yl)phenyl)pyridin-2-yl)ethyl)acrylamide; |
| 21 | (S)-N-((5-(4-(1-((5-cyclopropyl-1H- |
| | pyrazol-3-yl)amino)-1-oxopropan-2- |
| | yl)phenyl)-3-fluoropyridin-2-yl)methyl)acrylamide; |
| 22 | N-((6-(4-(1-((5-cyclopropyl-1H- |
| | pyrazol-3-yl)amino)-1-oxopropan-2- |
| | yl)phenyl)pyridin-3-yl)methyl)acrylamide; |
| 23 | N-(4-(1-((5-cyclopropyl-1H-pyrazol- |
| | 3-yl)amino)-1-oxopropan-2- |
| | yl)benzyl)acrylamide; |
| 24 | N-(4-(1-((5-cyclopropyl-1H-pyrazol- |
| | 3-yl)amino)-1-oxopropan-2- |
| | yl)benzyl)acrylamide (Isomer-1 of compound-23); |
| 25 | N-(4-(1-((5-cyclopropyl-1H-pyrazol- |
| | 3-yl)amino)-1-oxopropan-2- |
| | yl)benzyl)acrylamide (Isomer-2 of compound-23); |
| 26 | N-((5-(1-((5-cyclopropyl-1H-pyrazol- |
| | 3-yl)amino)-1-oxopropan-2-yl)pyridin-2- |
| | yl)methyl)acrylamide; |
| 27 | N-((5-(4-((5-cyclopropyl-1H-pyrazol- |
| | 3-yl)amino)-1-oxopropan-2- |
| | yl)phenyl)pyrimidin-2-yl)methyl)acrylamide; |
| 28 | N-((6′-(1-((5-cyclopropyl-1H-pyrazol- |
| | 3-yl)amino)-1-oxopropan-2-yl)-[3,3′- |
| | bipyridin]-6-yl)methyl)acrylamide; and |
| 29 | N-((5-(2-((5-cyclopropyl-1H-pyrazol- |
| | 3-yl)amino)-2-oxoethyl-[2,3′-bipyridin]- |
| | 6′-yl)methyl)acrylamide; |

or a pharmaceutically acceptable salt thereof, an N-oxide thereof or a stereoisomer thereof.

In certain embodiments, the present invention relates to a pharmaceutical composition, comprising at least one compound of formula (I), or a pharmaceutically acceptable salt or a stereoisomer thereof, and a pharmaceutically acceptable carrier or excipient.

In certain embodiments, the present invention relates to a compound or a pharmaceutically acceptable salt or a stereoisomer thereof, for use as a medicament.

Pharmaceutical Compositions

In certain embodiments, present invention provides a pharmaceutical composition comprising the compound of formula (I) or a pharmaceutically acceptable salt or a stereoisomer thereof, and at least one pharmaceutically acceptable carrier or excipient.

In certain embodiments, present invention provides a pharmaceutical composition comprising the compound of formula (I), for use in treating a subject suffering from a disease or condition associated with aberrant activity of CDK12/13.

In certain embodiments, the pharmaceutical composition of the present invention further comprises at least one agent selected from an anticancer agent, a chemotherapy agent, and an antiproliferative compound.

In certain embodiments, the present invention provides a pharmaceutical composition comprising a compound of formula (I), or a pharmaceutically acceptable salt thereof or a stereoisomer thereof as described herein and at least one pharmaceutically acceptable excipient (such as a pharmaceutically acceptable carrier or diluent). Preferably, the pharmaceutical composition comprises a therapeutically effective amount of at least one compound described herein. The compounds described in the present invention may be associated with a pharmaceutically acceptable excipient (such as a carrier or a diluent) or be diluted by a carrier or enclosed within a carrier which can be in the form of a capsule, sachet, paper or other container.

The compounds of the present invention may be used as single drug or as a pharmaceutical composition in which the compound is mixed with various pharmacologically acceptable materials.

The compounds of the invention are typically administered in the form of a pharmaceutical composition. Such compositions can be prepared using procedures well known in the pharmaceutical art and comprise at least one compound of the invention. The pharmaceutical composition of the present invention comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

A pharmaceutically acceptable carrier can contain pharmaceutically acceptable agents that act, for example, to stabilize, increase solubility or to increase the absorption of a compound such as a compound of the invention. Such pharmaceutically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a pharmaceutically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, pharmaceutically acceptable and metabolizable carriers that are relatively simple to make and administer.

The pharmaceutical composition can be administered by oral, parenteral or inhalation routes. Examples of the parenteral administration include administration by injection, percutaneous, transmucosal, intranasal and transpulmonary administrations.

Examples of suitable carriers include, but are not limited to, sterile water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the invention to the appropriate or desired site of action. Suitable routes of administration include, but are not limited to, oral, nasal, buccal, dermal, intradermal, transdermal, parenteral, rectal, subcutaneous, intravenous, intraurethral, intramuscular or topical.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers or finely divided solid carriers or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

Solid oral formulations include, but are not limited to, tablets, capsules (soft or hard gelatin), dragees (containing the active ingredient in powder or pellet form), troches and lozenges.

Solid oral formulations may contain, along with the active compound, lubricants, diluents, binding agents, disintegrating agents, wetting agents, preservatives, and in general, non-toxic, pharmacologically inactive substances used in pharmaceutical compositions.

The formulations may contain, lubricants, for ex., calcium stearate, magnesium stearate, stearic acid, talc, silica or polyethylene glycols; diluents, for ex., cellulose, corn starch, dextrose saccharose, lactose, potato starch, dry starch, sucrose, powdered sugar, mannitol, sorbitol, inositol, calcium carbonate, sodium carbonate, calcium phosphate, dicalcium phosphate and mixtures thereof; binding agents, for ex., Arabic gum, carboxymethylcellulose, gelatin methylcellulose, polyvinyl pyrrolidone or starches; disintegrating agents, for ex., alginic acid, alginates, starch or starch glycolate; wetting agents, for ex., lecithin, laurylsulphates or polysorbates; preservatives, for ex., antioxidants, chelating agents, antimicrobial preservatives, antifungal preservatives, antiprotozoan preservatives, alcohol preservatives.

Excipients such as cocoa butter, suppository waxes, colouring agents, coating agents, sweeteners, flavouring agents and perfuming agents may also be present in the composition.

Liquid formulations include, but are not limited to, syrups, emulsions, and sterile injectable liquids, such as suspensions or solutions.

Topical dosage forms of the compounds include ointments, pastes, creams, lotions, powders, solutions, eye or ear drops, impregnated dressings, and may contain appropriate conventional additives such as preservatives, solvents to assist drug penetration.

The pharmaceutical compositions of the present invention may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, and suitable pharmaceutical excipients can also be well used and adjusted by those skilled in the art. All changes and modifications are envisioned within the scope of the present invention.

According to one embodiment, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention, and their uses. Exemplary isotopes that can be incorporated in to compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds of the present inventions can generally be prepared by following procedures analogous to those disclosed in the schemes and/or in the examples herein below, by substituting an isotopically labeled reagent for a non-isotopically labeled reagent.

Methods of Treatment

In certain embodiments, the present invention provides compounds for use as a medicament.

In certain embodiments, the present invention provides compounds of formula (I) or a pharmaceutically acceptable salt, an N-oxide or a stereoisomer thereof, for use as a medicament.

In certain embodiments, the present invention provides compounds of formula (I) or a pharmaceutically acceptable salt, an N-oxide or a stereoisomer thereof, for use in the treatment of a cancer, an inflammatory disorder, an auto-inflammatory disorder or an infectious disease.

In certain embodiments, the invention provides the use of the compounds of the present invention in manufacturing of a medicament.

In certain embodiments, the invention provides a method of treating cancer or proliferative disorder, comprising administration of a therapeutically effective amount of a formula (I) or a pharmaceutically acceptable salt or a stereoisomer thereof.

In certain embodiments, the present invention provides methods for inhibiting growth of tumour cells and/or metastasis by administering a therapeutically effective amount of compound of formula (I) or a pharmaceutically acceptable salt or a stereoisomer thereof.

In certain embodiments, the present invention provides methods for treating cancer or proliferative disorder, by administering a therapeutically effective amount of compound of formula (I) or a pharmaceutically acceptable salt or a stereoisomer thereof.

According to yet another embodiment, the compounds of the present invention are useful in the treatment of proliferative diseases such as cancer, viral diseases, fungal diseases, neurological/neurodegenerative disorders, autoimmune diseases, inflammation, arthritis, anti-proliferative (e.g., ocular retinopathy), neuronal, alopecia and cardiovascular disease.

According to yet another embodiment, the present invention provides a compound of formula (I), for use in the treatment of a cancer.

According to yet another embodiment, the cancer is selected from the group consisting of a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate, and skin, including squamous cell carcinoma; hematopoietic tumors of lymphoid lineage, including leukemia, acute lymphoblastic leukemia, acute lymphocytic leukemia, Hodgkin's lymphoma, non-Hodgkin's lymphoma, B-cell lymphoma, T-cell lymphoma, hairy cell lymphoma, myeloma, mantle cell lymphoma, and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

According to yet another embodiment, the present invention provides a compound of formula (I) for use in the treatment of Myotonic Dystrophy type 1, Myotonic Dystrophy type 2, Fragile X associated tremor/ataxia syndrome, amylotrophic lateral sclerosis (ALS) and frontotemporal dementia, Huntington's disease like 2, Huntington's disease, several types of Spinocerebellar Ataxia, Dentatorubral-pallidoluysian atrophy and Spinal and Bulbar Muscular Atrophy.

In certain embodiments, the compounds of the present invention are selective CDK12/13 inhibitors (e.g., being selective for inhibition of CDK12/13 over CDK7).

In another embodiment, the present invention provides a method of inhibiting CDK12/13 in a subject, comprising administering to the subject a compound of formula (I).

In another embodiment, the present invention provides a method of selectively inhibiting CDK12/13 in a subject, comprising administering to the subject in need thereof a therapeutically effective amount of a compound of the present invention.

In another embodiment, the present invention provides a pharmaceutical composition for use in treating and/or preventing a disease and/or disorder associated with aberrant activity of CDK12/13.

In another embodiment, the present invention provides a pharmaceutical composition for use in treating a subject suffering from a disease or condition associated with aberrant activity of CDK12/13.

In another embodiment, the present invention provides pharmaceutical composition comprising the compound of formula (I), for use in treating a subject suffering from a disease or condition associated with aberrant activity of CDK12/13.

In another embodiment, the present invention provides a method of treating or preventing diseases and/or disorders or condition mediated by CDK12/13 in a subject comprising administering to the subject in need thereof a therapeutically effective amount of a compound or a pharmaceutically acceptable salt thereof, of the present invention.

According to the foregoing embodiment, the CDK12/13 mediated disorder or disease or condition is selected from the group consisting of a cancer, an inflammatory disorder, an auto-inflammatory disorder and an infectious disease.

According to the preceding embodiment, the cancer is selected from the group consisting of a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate, and skin, including squamous cell carcinoma; hematopoietic tumors of lymphoid lineage, including leukemia, acute lymphoblastic leukemia, acute lymphocytic leukemia, Hodgkin's lymphoma, non-Hodgkin's lymphoma, B-cell lymphoma, T- cell lymphoma, hairy cell lymphoma, myeloma, mantle cell lymphoma, and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system,including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

In yet another embodiment, the compounds as disclosed in the present invention are formulated for pharmaceutical administration.

Yet another embodiment of the present invention provides use of compounds of the present invention in the treatment and prevention of diseases or disorder associated with the aberrant activity of CDK12/13.

Yet another embodiment of the present invention provides use of compounds of the present invention in the treatment of a cancer, an inflammatory disorder, an auto-inflammatory disorder or an infectious disease.

Yet another embodiment of the present invention provides the use of a compound or a pharmaceutically acceptable salt thereof, in treating and/or preventing a disease for which the symptoms thereof are treated, improved, diminished and/or prevented by selective inhibition of CDK12/13.

According to yet another embodiment, the CDK12/13 mediated disorder and/or disease or condition is proliferative disease or disorder or condition.

In yet another embodiment, the diseases and/or disorder mediated by CDK12/13 is selected from the group consisting of a cancer, an inflammatory disorder, an auto-inflammatory disorder and an infectious disease.

In other embodiments, the proliferative disease to be treated or prevented using the compounds of formula (I) will typically be associated with aberrant activity of CDK12/13.

In certain embodiments, CDK12/13 refers to CDK 12 or CDK 13 or CDK 12 and CDK13.

According to yet another embodiment, the disorder or condition mediated by CDK12/13 is Myotonic Dystrophy type 1, Myotonic Dystrophy type 2, Fragile X associated tremor/ataxia syndrome, amylotrophic lateral sclerosis (ALS) and frontotemporal dementia, Huntington's disease like 2, Huntington's disease, several types of Spinocerebellar Ataxia, Dentatorubral-pallidoluysian atrophy and Spinal and Bulbar Muscular Atrophy.

According to yet another embodiment, the diseases and/or disorder mediated by CDK12/13 is Myotonic dystrophy.

According to yet another embodiment, the compounds of the present invention are useful in the treatment of Myotonic dystrophy.

According to yet another embodiment, the present invention provides a method of treating Myotonic dystrophy by administering a therapeutically effective amount of a compound of formula (I).

According to yet another embodiment, the present invention provides compounds of formula (I) in the manufacture of a medicament for treating Myotonic dystrophy.

According to yet another embodiment, the subject is a mammal including human.

According to yet another embodiment, the present invention provides compounds or pharmaceutically acceptable salts or stereoisomers thereof, for use as a medicament.

According to yet another embodiment, the invention provides the use of the compounds of the present invention in the manufacture of a medicament.

According to yet another embodiment, the present invention provides compounds or pharmaceutically acceptable salts or stereoisomers thereof, for use in the treatment of cancer.

According to yet another embodiment, the present invention provides compounds or pharmaceutically acceptable salts or stereoisomers thereof, for use in the treatment of an inflammatory disorder, an auto-inflammatory disorder or an infectious disease.

According to yet another embodiment, the invention provides the use of the compounds of the present invention in the manufacture of a medicament for the treatment of diseases and/or disorder associated with the aberrant activity of CDK12/13.

In yet another embodiment, the invention provides the use of the compounds of the present invention in the manufacture of a medicament for the treatment of cancer.

In yet another embodiment, the invention provides the use of the compounds of the present invention in the manufacture of a medicament for the treatment of an inflammatory disorder, an auto-inflammatory disorder or an infectious disease.

According to yet another embodiment, the present invention provides compounds for use as a medicament for treating a subject suffering from diseases and/or disorder associated with aberrant activity of CDK12/13.

According to yet another embodiment, the present invention comprises administering to the subject in need thereof a therapeutically effective amount of a compound of the present invention along with one or more additional chemotherapeutic agents independently selected from anti-proliferative agents, anti-cancer agents, immunosuppressant agents and pain-relieving agents.

In yet another embodiment, the present invention provides a method comprising an additional step of administering to the subject in need thereof one or more additional chemotherapeutic agents independently selected from anti-proliferative agents, anti-cancer agents, immunosuppressant agents and pain-relieving agents.

According to yet another embodiment, the chemotherapeutic agents are selected from, but not limited to, CPT-11, captothecin derivatives, taxane, taxane derivatives, encapsulated taxanes, anthracyclin glycosides, for ex., doxorubicin, idarubicin, epirubicin, etoposide, navelbine, vinblastine, carboplatin, cisplatin, estramustine, celecoxib, Sugen SU-5416, Sugen SU-6668, Herceptin, optionally within liposomal formulations thereof.

According to yet another embodiment, the anti-cancer agents are selected from, but not limited to, Atezolizumab, Avelumab, Bevacizumab, Cetuximab, ipilimumab, nivolumab, Obinutuzumab, Panitumumab, Pembrolizumab, Pertuzumab, Vinblastine, Vincristine, Zoladex, Abemaciclib, palbociclib, Ribociclib, Kymriah, Letrozole, Avappritinib, Bosutinib, Ceritinib, Crizotinib, Dasatinib, Erlotinib Hydrochloride, Gefitinib, Imatinib Mesylate,

Ibrutinib, Sunitinib, and the like.

In certain other embodiments, the chemotherapeutic agent is methotrexate, doxorubicin hydrochloride, chlorambucil, nelarabine, ofatumumab, bosutimab, busulfan, alemtiizumab, daunorubicin hydrochloride, cyclophosphamide, clofarabine, cytarabine, cyclophosphamide, Asparaginase Erwinia Chrysanthemi, fiudarabine phosphate, obinutuzumab, ponatinib hydrochloride, ibrutinib, vincristine sulfate liposome, mitoxantrone hydrochloride, mechlorethamine hydrochloride, Pegaspargase, mercaptopurine, Rubidomycin daunorubicin hydrochloride, omacetaxine mepesuccinate, cytarabine, nilotinib, bendamustine hydrochloride, arsenic trioxide, vincristine sulfate, idelalisib, or a combination thereof.

In certain embodiments, the additional chemotherapeutic agent is an anti-lymphoma agent. In certain embodiments, the additional chemotherapeutic agent is brentuximab vedotin, doxorubicin hydrochloride, nelarabine, tositumomab, bleomycin, dacarbazine, pralatrexate, recombinant interferon alfa-2b, romidepsin, Lomustine, procarbazine hydrochloride, plerixafor, mechlorethamine hydrochloride, lenalidomide, rituximab, bendamustine hydrochloride, vinblastine sulfate, bortezomib, vincristine sulfate, ibritumomab tiuxetan, orinostat, or a combination thereof.

In certain embodiments, the additional chemotherapeutic agent is ABITREXATE, ABRAXANE, ADRIAMYCIN PFS, ADRUCIL, AFINITOR, AFINITOR DISPERZ, ALDARA, ALIMTA, AREDIA, ARIMIDEX, AROMASIN, AVASTIN, BECENUM, BICNIJ, BLENOXANE, CAMPTOSAR, CAPDX, CAPRELSA, C ARBOPL ATTN—TAXOL, CARMUBRIS, CASODEX, CERUBIDLNE, CERVARTX, CLAFEN, COMETRIQ, COSMEGEN, CYFOS, CYRAMZA, CYTOSAR-U, CYTOXAN, DACOGEN, DEGARELIX, DOXIL, DOXORUBICIN HYDROCHLORIDE, EFUDEX, ELLENCE, ELOXATIN, ERBITUX, ERIVEDGE, ETOPOPHOS, EVACET, FARESTON, FASLODEX, FEMARA, FLUOROPLEX, ACIZUMAB, FOLFIRI-CETUXIMAB, FOLFIRINOX, FOLFOX, GARDASIL, GEMCITABINE-OXALIPLATIN, GEMZAR, GILOTRIF, GLEEVEC, GLIADEL, GLIADEL WAFER, HERCEPTIN, HYCAMTIN, IFOSFAMIDUM, INLYTA, KEYTRUDA KYPROLIS, LIPODOX, LUPRON DEPOT, MEGACE, METHAZOLASTONE, MITOXANTRONE HYDROCHLORIDE, MITOZYTREX, MOZOBIL, MUSTARGEN, MUTAMYCIN, MYLOSAR, NAVELBINE, NEXAVAR NOLVADEX, PARAPLATIN, PLATINOL, PROLEUKIN, STIVARGA, TAFINLAR, TEMODAR, THALOMID, TOPOSAR, TORTSEL, TRISENOX, VECTIBIX

VIADUR VIDAZA, ZALTRAP, ZOLADEX, ZOMETA, ZYKADI A, ZYTIGA, or a combination thereof.

The method(s) of treatment of the present invention comprises administering a safe and effective amount of a compound according to formula (I) or a pharmaceutically acceptable salt thereof to a patient (particularly a human) in need thereof.

Compounds of the present invention are indicated both in the therapeutic and/or prophylactic treatment of the above-mentioned conditions. For the above-mentioned therapeutic uses the dosage administered will, of course, vary with the compound employed, the mode of administration, the treatment desired and the disorder or disease indicated.

Definitions:

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used herein, the term “optionally substituted” refers to replacement of one or more hydrogen radicals in a given structure with a radical of a specified substituent including, but not limited to: halo, alkyl, alkenyl, alkynyl, aryl, heterocyclyl, thiol, alkylthio, arylthio, alkylthioalkyl, arylthioalkyl, alkyl sulfonyl, alkylsulfonylalkyl, aryl sulfonyl alkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonyl alkyl, acyl, aralkoxycarbonyl, carboxylic acid, sulfonic acid, sulfonyl, phosphonic acid, cycloalkyl, heteroaryl, and aliphatic. It is understood that the substituent may be further substituted.

As used herein, unless otherwise defined the term “alkyl” alone or in combination with other term(s) means saturated aliphatic hydrocarbon chain, including C₁-C₁₀ straight or C₁-C₁₀ branched alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include but are not limited to methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

As used herein, the term “halo” or “halogen” alone or in combination with other term(s) means fluorine, chlorine, bromine or iodine.

As used herein, the term “alkoxy” refers to an alkyl group as hereinbefore defined, bonded to an oxygen atom that is attached to a core structure. Preferably, alkoxy groups have one to six carbon atoms. Alkoxy may be represented as alkyl-O—or —O-alkyl, where alkyl groups are as defined above. Exemplary alkoxy groups include but are not limited to methoxy, ethoxy, propoxy, isopropoxy, butoxy, isobutoxy, tert-butoxy, pentoxy, 3-methyl butoxy and the like. An alkoxy group can be optionally substituted with one or more suitable groups.

The term “heteroatom” as used herein designates a sulfur, a nitrogen or an oxygen atom.

As used herein, the term “heterocycloalkyl” refers to a non-aromatic, saturated or partially saturated, bridged bicyclic, spirocyclic, monocyclic or polycyclic ring system of 3 to 15 member having at least one heteroatom or heterogroup selected from O, N, S, S(O), S(O)₂, NH and C(O) with the remaining ring atoms being independently selected from the group consisting of carbon, oxygen, nitrogen, and sulfur. The term “heterocycloalkyl” also refers to the bridged bicyclic ring system having at least one heteroatom or hetero group selected from O, N, S, S(O), S(O)₂, NH and C(O). Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, aza-bicyclooctanyl, azocinyl, chromanyl, isochromanyl xanthenyl, 2-oxa-6-azaspiro[3.3]heptanyl, 3-oxa-8-azabicyclo[3.2.1]octanyl and N-oxides thereof. Attachment of a heterocycloalkyl substituent can occur via either a carbon atom or a heteroatom. A heterocycloalkyl group can be optionally further substituted.

As used herein the term “cycloalkyl” alone or in combination with other term(s) means C₃-C₁₀ saturated cyclic hydrocarbon ring. A cycloalkyl may be a single ring, which typically contains from 3 to 7 carbon ring atoms. Examples of single-ring cycloalkyls include but are not limited to cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused and spirocyclic carbocyclyls and the like.

As used herein, the term “aryl” is optionally substituted monocyclic, bicyclic or polycyclic aromatic hydrocarbon ring system of about 6 to 14 carbon atoms. Examples of a C₆-C₁₄ aryl groups include, but are not limited to phenyl, naphthyl, biphenyl, anthryl, tetrahydronaphthyl, fluorenyl, indanyl, biphenylenyl and acenaphthyl. Aryl group can be optionally substituted with one or more suitable groups.

As used herein, the term “heteroaryl” alone or in combination with other term(s) means a completely unsaturated ring system containing a total of 5 to 14 ring atoms. At least one of the ring atoms is a heteroatom (i.e., oxygen, nitrogen, or sulfur), with the remaining ring atoms/groups being independently selected from carbon, oxygen, nitrogen or sulfur. A heteroaryl may be a single-ring (monocyclic) or multiple rings (bicyclic, tricyclic or polycyclic) fused together or linked covalently. Preferably, “heteroaryl” is a 5- to 6-membered ring. The rings may contain from 1 to 4 additional heteroatoms selected from N, O and S, wherein the N atom is optionally quarternized. Any suitable ring position of the heteroaryl moiety may be covalently linked to the defined chemical structure. Examples of “heteroaryl” include but are not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, 3-fluoropyridyl, pyrimidinyl, pyrazinyl, pyridazinyl, benzoxazolyl, benzisoxazolyl;

benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, a-carbolinyl, indolizinyl, benzoisothiazolyl, benzoxazolyl, pyrrolopyridyl, furopyridinyl, purinyl, benzothiadiazolyl, benzooxadiazolyl, benzotriazolyl, benzotriadiazolyl, carbazolyl, dibenzothienyl, acridinyl and the like. Heteroaryl group may be optionally further substituted.

As used herein, the term “heterocyclyl” or “heterocyclic” alone or in combination with other term(s) includes both “heterocycloalkyl” and “heteroaryl” groups which are as defined above.

Certain of the compounds disclosed herein can exist as N-oxides. For example, it is known that the pyrazoles can form N-oxides on treatment with a suitable oxidizing agent. Similarly, it is known that the pyridine ring nitrogen can be oxidized on treatment with a suitable oxidizing agent to form an N-oxide.

As used herein, the term “compound(s)” comprises the compounds disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or its pharmaceutically acceptable salt; and a conventional pharmaceutically acceptable carrier.

The pharmaceutical composition(s) of the present invention can be administered orally, for example in the form of tablets, coated tablets, pills, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermals, in the form of patches, or in other ways, for example in the form of aerosols or nasal sprays.

The pharmaceutical composition(s) usually contain(s) about 1% to 99%, for example, about 5% to 75%, or from about 10% to about 30% by weight of the compound of formula (I) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

As used herein, the term “treat”, “treating” and “treatment” refer to any treatment of a disease in a mammal, including: (a) Inhibiting the disease, i.e., slowing or arresting the development of clinical symptoms; and/or (b) relieving the disease, i.e., causing the regression of clinical symptoms and/or (c) alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal, and most preferably a human.

As used herein, the term “therapeutically effective amount” refers to that amount of a compound of formula (I) or a pharmaceutically acceptable salt or a stereoisomer thereof; or a composition comprising the compound of formula (I) or a pharmaceutically acceptable salt or a stereoisomer thereof, effective in producing the desired therapeutic response in a particular patient suffering from a diseases or disorder, in particular their use in diseases or disorder associated with cancer. Particularly, the term “therapeutically effective amount” includes the amount of the compound of formula (I) or a pharmaceutically acceptable salt or a stereoisomer thereof, when administered, that induces a positive modification in the disease or disorder to be treated or is sufficient to prevent development of, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

“Pharmaceutically acceptable salt” refers to a product obtained by reaction of the compound of the present invention with a suitable acid or a base. Pharmaceutically acceptable salts of the compounds of this invention include those derived from suitable inorganic and organic acids and bases. Such salts include: acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methane sulfonic acid, ethane sulfonic acid, 1,2-ethane-disulfonic acid, 2-hydroxyethanesulfonic acid, benzene sulfonic acid, 4-chlorobenzenesulfonic acid, 2-naphthalenesulfonic acid, 4-toluenesulfonic acid, camphor sulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxyl naphthoic acid, salicylic acid, stearic acid, muconic acid, and the like. Certain compounds of the invention (compounds of formula (I)) can form pharmaceutically acceptable salts with various organic bases such as lysine, arginine, guanidine, diethanolamine or metformin.

The term “stereoisomers” refers to any enantiomers, diastereoisomers, or geometrical isomers of the compounds of Formula (I), or any subformulae, e.g., formulae (IA) to (IK), or compounds as disclosed herein, wherever they are chiral or when they bear one or more double bonds. When the compounds of the Formula (I) and related formulae are chiral, they can exist in racemic or in optically active form. It should be understood that the invention encompasses all stereochemical isomeric forms, including diastereomeric, enantiomeric and epimeric forms, (R) and (S) isomers, as well as d-isomers and l-isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E) and zusammen (Z) isomers as well as the appropriate mixtures thereof.

### Experimental Section

Development of CDK12/13 Specific Inhibitor

The primary aim of the present invention was to improve the CDK12/13 specificity of the compounds disclosed in WO 2016/193939. Surprisingly, it was found that by altering the substituent relationship of the central aromatic ring from a 1,3 relationship (i.e. meta substituted) to a 1,4 relationship (i.e. para substituted), the selectivity of the compound for the CDK12/13 receptor was greatly improved.

For example, modifying compound-A (compound-74 in WO 2016/193939) by changing the substituent relationship of the central aromatic moiety from meta to para (thereby producing compound-8 of the present invention) resulted in a significant loss of binding at the CDK7 receptor whilst maintaining excellent binding at the CDK12/13 receptor.

| | | Compound-A |
|---|---|---|
| 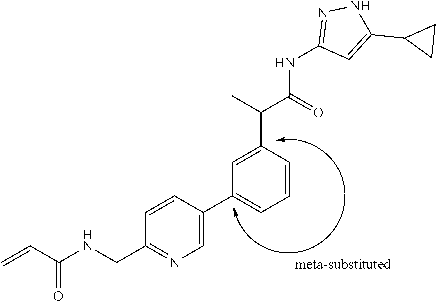 [SMILES: C=CC(=O)NCc1ccc(-c2cccc(C(C)C(=O)Nc3cc(C4CC4)[nH]n3)c2)cn1] | | |
| | | Compound-8 |
| 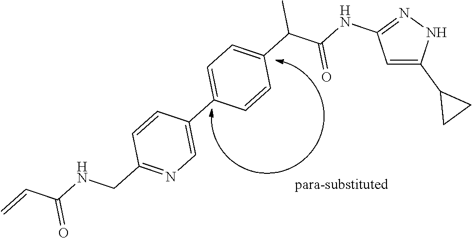 [SMILES: C=CC(=O)NCc1ccc(-c2ccc(C(C)C(=O)Nc3cc(C4CC4)[nH]n3)cc2)cn1] | | |
| Parameter | Compound A | Compound-8 |
| Jurkat Cells, CDK12 Target Occ₅₀ (μM) | 0.23 (n = 4) | 0.022 |
| Jurkat Cells, CDK7 Target Occ₅₀ (μM) | 0.61 (n = 2) (2×) | >>2 |

Accordingly, reducing the binding affinity of the compound for CDK7 whilst maintaining or improving the binding affinity for CDK12/13 results in an inhibitor that selectively targets CDK12/13 over CDK7. The following sets out the synthesis and evaluation of further exemplary CDK12/13 inhibitors of the invention having a central aromatic group with the 1,4 substituent relationship.

### General Modes of Preparation:

Following general guidelines applies to all experimental procedures described here. Until otherwise stated, experiments are performed under positive pressure of nitrogen, temperature describes are the external temperature (i.e. oil bath temperature). Reagents and solvents received from vendors are used as such without any further drying or purification. Molarities mentioned here for reagents in solutions are approximate as they were not verified by a prior titration with a standard. All reactions were stirred under magnetic stir bar. Cooling to temperatures below 0° C., was done by using a bath of either acetone/dry ice or wet ice/salts. Magnesium sulfate and sodium sulfate were used as solvent drying agent after reaction work up and are interchangeable. Removing of solvents under reduced pressure or under vacuum means distilling of solvents in rotary evaporator.

Compounds of this invention may be made by synthetic chemical processes, examples of which are shown herein. It is meant to be understood that the order of the steps in the processes may be varied, that reagents, solvents and reaction conditions may be substituted for those specifically mentioned and that vulnerable moieties may be protected and deprotected, as necessary.

The specifics of the process for preparing compounds of the present invention are detailed in the experimental section.

The present invention shall be illustrated by means of some examples, which are not construed to be viewed as limiting the scope of the invention.

Unless otherwise stated, work-up includes distribution of the reaction mixture between the organic and aqueous phases, separation of layers and drying the organic layer over anhydrous sodium sulphate, filtration and evaporation of the solvent. Purification, unless otherwise mentioned, includes purification by silica gel chromatographic techniques, generally using ethyl acetate/petroleum ether mixture of a suitable polarity as the mobile phase.

Analysis for the compounds of the present invention unless mentioned, was conducted in the general methods well known to the person skilled in the art. The absolute stereoconfiguration of the chiral compounds as disclosed herein can be determined by methods known in the art. Having described the invention with reference to certain preferred embodiments, other embodiments will become apparent to one skilled in the art from consideration of the specification. The invention is further defined by reference to the following examples, describing in detail the analysis of the compounds of the invention.

It will be apparent to those skilled in the art that many modifications, both to materials and methods, may be practiced without departing from the scope of the invention. Some of the intermediates were taken to next step based on TLC results, without further characterization, unless otherwise specified.

The following abbreviations refer respectively to the definitions herein: LDA (Lithium diisopropylamide); K₂CO₃ (Potassium carbonate); KOAc (Potassium acetate); EtOH (Ethanol); Prep TLC (Preparative Thin layer Chromatography); rt (Retention time); RT (Room temperature); DMF (Dimethylformamide); h (hour); NaOH (Sodium hydroxide); THF (tetrahydrofuran); HATU (1-[Bis(dimethylamino)methylene]-1H-1,2,3 -triazolo[4, 5-b)]pyridinium 3-oxid-hexafluorophosphate); LC-MS (Liquid chromatography mass spectroscopy); HCl (Hydrochloric acid); DCM, CH₂Cl₂ (Dichloromethane); TFA (Trifluoroacetic acid); TLC (Thin layer chromatography); DIPEA (Diisopropyl Ethyl amine); Na₂SO₄ (Sodium sulphate); ACN/CH₃CN (Acetonitrile); TCP (Tricyclohexylphosphine); NaBH₄ (Sodium borohydride); (COCl)₂ (Oxalyl chloride); PdCl₂(dppf)-DCM (1,1′-Bis(diphenylphosphino)ferrocene dichloropalladium(II).dichloromethane complex); Pd(dba)₃ (Tris(dibenzylideneacetone)dipalladium(O); Bpin₂ (Bis(pinacolato)diboron); Pd(PPh₃)₄ (Tetrakis[triphenylphosphine]palladium(O)); MeOH (Methanol); NiCl₂.6H₂O (Nickel(II) chloride hexahydrate); DMSO-d₆ (Dimethyl sulfoxide-d); Boc₂O (Ditert-butyl dicarbonate); HPLC (High pressure liquid chromatography); NaHCO₃ (Sodium bicarbonate); TEA (triethyl amine), Cs₂CO₃ (Cesium carbonate); MHz (megahertz); s (singlet); m (multiplet); and d (doublet).

### Synthesis of Intermediates:

Intermediate-1: Synthesis of (E)-4-morpholino-N-(5-(4,4,5,5-tetramethyl-1,3,2-dioxaborolan-2-yl)pyridin-2-yl)but-2-enamide

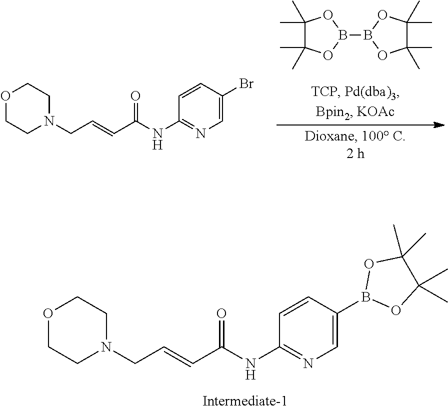

To a degassed dioxane (1.25 L) solvent was added Tricyclohexylphosphine (8.5 g, 0.03 mol) and stirred mixture till it dissolves. After clear solution Pd(dba)₃ (14 g, 0.015 mol) was added followed by potassium acetate (60.12 g, 0.613 mol), (E)-N-(5-bromopyridin-2-yl)-4-morpholinobut-2-enamide (100 g, 0.30 mol) (synthesis carried out as described in reference WO2016/193939 A1) and Bis(pinacolato)diboron (116 g, 0.46 mol) under argon purging, reaction mixture was stirred at 100° C. for 2 h, then cooled to room temperature and filtered on celite bed, filtrate was evaporated to dryness and added 50% Pentane-Ether (500 mL) (off white precipitation observed). Mixture was stirred for 10 min and filtered under vacuum, washed with pentane (250 mL) and dried to get off white solid (100 g). Crude product was re-dissolved in 10% MeOH in MDC (1 L) and 10 g charcoal added, refluxed for 30 min, filtered on celite bed and evaporated to dryness to get off white solid (80 g, 70% yield). LCMS: m/z=291.9 (M+H)⁺. (Boronic acid mass).

Intermediate-2: Synthesis of (E)-N-((5-bromopyridin-2-yl)methyl)-4-morpholinobut-2-enamide

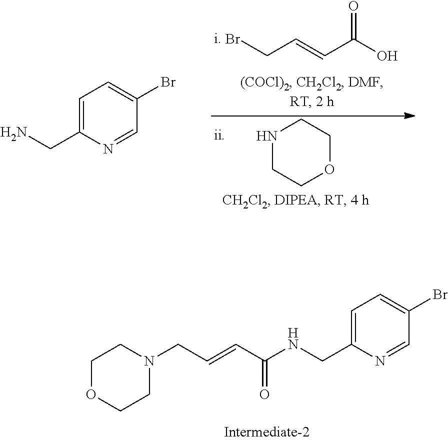 [SMILES: O=C(O)/C=C/CBr ; NCc1ccc(Br)cn1 ; C1COCCN1 ; O=C(/C=C/CN1CCOCC1)NCc1ccc(Br)cn1]

(E)-4-bromobut-2-enoic acid (445 mg, 2.70 mmol) was taken in DCM (5 mL) with catalytic amount of DMF followed by the addition of oxalyl chloride (0.4 mL). The reaction mass was allowed to stir for 2 h at RT, evaporated the solvent under vacuum. The residue was dissolved in DCM (5 mL) and was added to the precooled solution of (5-bromopyridin-2-yl)methanamine (500 mg, 2.70 mmol) in DCM (5 mL) and DIPEA (0.690 mL, 5.4 mmol) at 0° C. The resulting reaction mixture was stirred for 10 min at 0° C. and after completion of the reaction, morpholine (704 mg, 8.1 mmol) was added and then allowed to stir at room temperature for 4 h. The reaction mixture was quenched with ice cold water and diluted with DCM. The aqueous layer was separated and extracted with DCM (2×25 mL). The combined organic phase was washed with brine, dried over Na₂SO₄, filtered and concentrated under reduced pressure and purified the residue with silica column chromatography by eluting with 10% methanol-DCM to afford the title compound (500 mg, 54%). LCMS: m/z=341 (M+H)⁺.

Intermediate-3: Synthesis of N-((5-bromo-3-fluoropyridin-2-yl)methyl)acrylamide

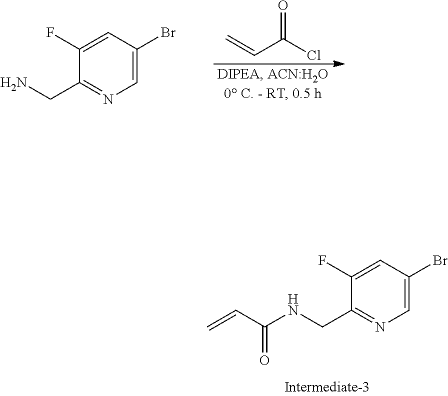

To a solution of (5-bromo-3-fluoropyridin-2-yl)methanamine hydrochloride salt (0.5 g, 2.10 mmol) in ACN (20 mL) were added water (2 mL), DIPEA (0.54 mL, 4.20 mmol) and acryloyl chloride (0.201 g, 2.10 mmol) at 0° C. After 30 min, the reaction mixture was quenched with ice-water and diluted with EtoAc. The aqueous layer was separated and extracted with EtOAc (2×25 mL). The combined organic phase was washed with brine, dried over Na₂SO₄, filtered and concentrated. The crude residue was purified by silica column chromatography by eluting with 0-5% MeOH-DCM to afford the title compound (0.3g, 47%). LCMS: m/z=261 (M+H)⁺, HPLC: 95%, rt: 4.6min.

Intermediate-4: Synthesis of tert-butyl 5-(2-(4-bromophenyl)propanamido)-3-cyclopropyl-1H-pyrazole-1-carboxylate

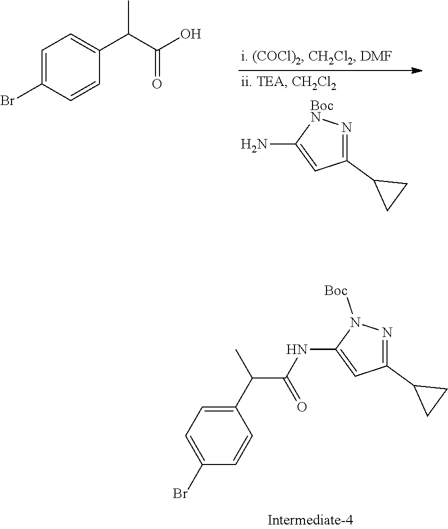 [SMILES: CC(C(=O)O)c1ccc(Br)cc1 ; CC(C)(C)OC(=O)n1nc(C2CC2)cc1N ; CC(C(=O)Nc1cc(C2CC2)nn1C(=O)OC(C)(C)C)c1ccc(Br)cc1]

2-(4-bromophenyl)propanoic acid (1 g, 4.36 mmol) (synthesis carried out as described in reference WO2016/193939 A1) was taken in DCM (10 mL) at 0° C. with catalytic amount of DMF and added oxalyl chloride (0.82 g, 6.54 mmol), allowed to stir the reaction mass at room temperature for 1.5 h. Concentrated the reaction mass under vacuum and the residue was dissolved in dry DCM (5 mL) and added to the cooled solution of tert-butyl 5-amino-3-cyclopropyl-1H-pyrazole-1-carboxylate (0.97 g, 4.36 mmol) (synthesis carried out as described in reference Tetrahedron Letters,2005, vol. 46, #6 p. 933-935), TEA (1.1 mL, 8.72 mmol) in DCM (10 mL) at 0° C. The resultant reaction mass was stirred at room temperature for 2 h, and diluted with DCM then washed with water followed by brine solution. The organic layer was dried over anhydrous sodium sulphate, concentrated under vacuum and crude compound was purified with silica column chromatography by eluting with 10%-30% hexane-ethyl acetate to afford the title compound (1 g, 53%). LCMS: m/z=336.1 (M-Boc+2).

Intermediate-5: Synthesis of tert-butyl 5-(2-(6-chloropyridin-3-yl)propanamido)-3-cyclopropyl-1H-pyrazole-1-carboxylate

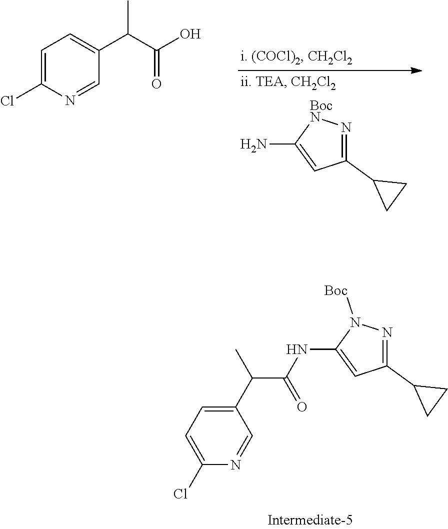 [SMILES: CC(C(=O)O)c1ccc(Cl)nc1 ; CC(C)(C)OC(=O)n1nc(C2CC2)cc1N ; CC(C(=O)Nc1cc(C2CC2)nn1C(=O)OC(C)(C)C)c1ccc(Cl)nc1]

The Intermediate-5 was prepared by a procedure similar to the one described in

Intermediate-4 by using 2-(6-chloropyridin-3-yl)propanoic acid as starting material. ¹HNMR (CDCl3, 400MHz): δ 10.36 (s, 1H), 8.37 (d, 1H), 7.28-7.09 (m, 1H), 7.34-7.31 (m, 1H), 6.37 (s, 1H), 3.77-3.69 (m, 1H), 1.99-1.93 (m, 1H), 1.63 (d, 3H), 1.53 (s, 9H), 0.96-0.91 (m, 2H), 0.95-0.70 (m, 2H). LCMS: m/z=391.9 (M+H)⁺.

Intermediate-6: Synthesis of tert-butyl (S)-5-(2-(4-bromophenyl)propanamido)-3-cyclopropyl-1H-pyrazole-1-carboxylate

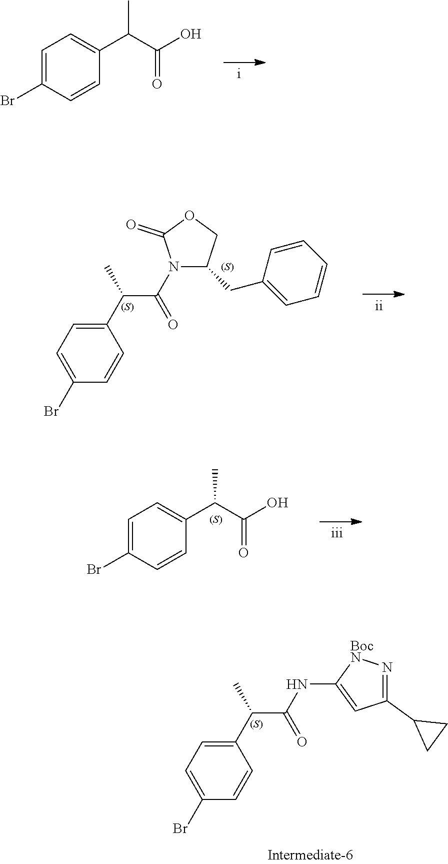

Reagents and conditions: i. a) Pivolyl chloride, NMM, THF; b) n-BuLi, THF; ii. H₂O₂, LiOH, THF, 0° C-RT; iii. a) (COCl)₂, CH₂Cl₂, DMF; b) Proton sponge, Toluene, 0° C-RT.

Step-i: Synthesis of (S)-4-benzyl-3-((S)-2-(4-bromophenyl)propanoyl)oxazolidin-2-one

To a solution of 2-(4-bromophenyl) propanoic acid (65 g, 0.28 mol) in THF (500 mL), N-methylmorpholine (33 mL, 0.3 mol) and Pivolylchloride (37 mL, 0.3 mol) were added sequentially at 0° C. Reaction mixture was warm to room temperature and stirred for 3 h. Meanwhile in another RBF n-BuLi (2.5 M in Hexane, 116 mL,0.28 mol) was added to solution of (S)-4-Benzyl-2-oxazolidinone (50 g, 0.28 mol) in THF at -78° C. and stirred for 1 h. Earlier made reaction mixture was added to this mixture slowly at −78° C. for 0.5 h and stirred for 1 h at same temperature. Reaction was quenched with saturated NH₄C1 solution and organic phase was separated from aqueous layer. Aqueous layer was further extracted with ethyl acetate and combined organic layer was dried over anhydrous sodium sulphate and concentrated under reduced pressure to afford crude compound. Crude product having a mixture of (S)-4-benzyl-3-((S)-2-(4-bromophenyl)propanoyl)oxazolidin-2-one and (S)-4-benzyl-3-((R)-2-(4-bromophenyl)propanoyl)oxazolidin-2-one were purified with silica column chromatography by eluting with 97%-30% hexane-ethyl acetate to isolate desired product (S)-4-benzyl-3-((S)-2-(4-bromophenyl)propanoyl)oxazolidin-2-one (42 g, 38.5%), LCMS: m/z=386 (M−H), Chiral HPLC: 98.84%, rt : 7.54 min.

Step-ii: Synthesis of (S)-2-(4-bromophenyl)propanoic acid

LiOH (9.3 g, 0.22 mol) was dissolved in water (150 mL) and added to solution of (S)-4-benzyl-3-((S)-2-(4-bromophenyl)propanoyl)oxazolidin-2-one (42 g, 0.11 mol) in THF (250 mL) and H₂O₂ (30% w/v solution, 40 mL, 0.33 mol) at 0° C. Resultant mixture was brought to room temperature and stir for 2 h. Reaction mass was quenched with saturated Na₂SO₃ solution and diluted with ether. Organic phase was separated from aqueous layer and washed with ethyl acetate twice. Separated aqueous layer was acidified using 2N HC1 and extracted with DCM twice, washed with brine, dried over anhydrous sodium sulphate and concentrated under vacuum to get crude compound. Crude was further purified by using ethylacetate:hexane (3:97) mixture as mobile phase to afford pure title compound (17 g, 67%). ¹H NMR (CDCl₃, 400MHz): δ 7.26-7.01 (m, 1H), 7.47-7.44 (m, 1H), 3.71-3.69 (m, 1H), 1.51 (d, 3H), LCMS: m/z=228.9 (M-H)⁺, HPLC: 99.60%, Chiral HPLC; 99.33%, rt: 8.91 min.

Step-iii: Synthesis of tert-butyl (S)-5-(2-(4-bromophenyl)propanamido)-3-cyclopropyl-1H-pyrazole-1-carboxylate

(S)-2-(4-bromophenyl) propanoic acid (16.5 g, 73 mmol) was dissolved in dry DCM (100 mL) and added oxalyl chloride (11.1 g, 88 mmol) at 0° C. followed by dropwise addition of catalytic amount of DMF and stirred for 30 min at the same temperature. Reaction mass was warm to room temperature and stirred for another 2 h. Excess of solvent and oxalylchloride was evaporated under normal reduce pressure. Residue was re-dissolved in toluene and added to the solution of tert-butyl 3-amino-5-cyclopropyl-1H-pyrazole-1-carboxylate (16.5 g,73 mmol) and 1,8-Bis(dimethylamino)naphthalene (Proton sponge) (15.6 g, 73 mmol) in toluene (250 mL) at 0° C. Reaction mixture was stirred for 2 h then solvent was removed under reduce pressure and residue was dissolved in DCM, washed with water, dried over anhydrous sodium sulphate and evaporated to get brown residue. Crude compound was further purified by column chromatography (10% of Ethyl acetate in hexane) to get the pure compound (15 g, 47%). LCMS: m/z=434.05 (M+H)⁺, HPLC: 96.10%, Chiral HPLC; 98.84%, rt: 6.64 min.

Intermediate-7: Synthesis of tert-butyl 5-(2-(6-chloropyridin-3-yl)acetamido)-3-cyclopropyl-1H-pyrazole-1-carboxylate.

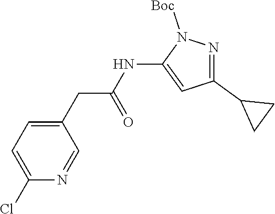 [SMILES: CC(C)(C)OC(=O)n1nc(C2CC2)cc1NC(=O)Cc1ccc(Cl)nc1]

The Intermediate-7 was prepared by a procedure similar to the one described in Intermediate-5 with appropriate variations in reactants. The characterization data of the compound is summarized herein below:

Characterization Data: ¹H NMR (DMSO-d₆, 400MHz): δ 10.20 (s, 1H), 8.34-8.33 (m, 1H), 7.81-7.78 (m, 1H), 7.51-7.48 (m, 1H), 6.31 (s, 1H), 3.89 (s, 2H), 1.90-1.85 (m, 1H), 1.56 (s, 9H), 0.93-0.86 (m, 2H), 0.68-0.63 (m, 2H). LCMS: m/z=377.10 (M+H)⁺.

### EXAMPLES

The present invention is further exemplified, but not limited, by the following examples that illustrate the preparation of compounds according to the invention.

### Example-1

### Synthesis of N-(4′-(1-((5-cyclopropyl-1H-pyrazol-3-yl)amino)-1-oxopropan-2-yl)-[1,1′-biphenyl]-4-yl)acrylamide (Compound -1)

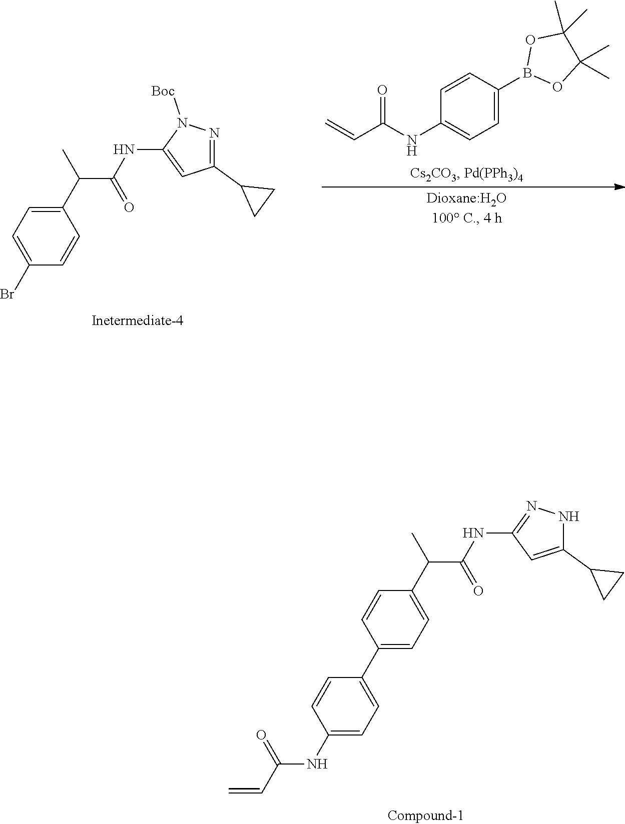 [SMILES: CC(C(=O)Nc1cc(C2CC2)nn1C(=O)OC(C)(C)C)c1ccc(Br)cc1 ; C=CC(=O)Nc1ccc(B2OC(C)(C)C(C)(C)O2)cc1 ; C=CC(=O)Nc1ccc(-c2ccc(C(C)C(=O)Nc3cc(C4CC4)[nH]n3)cc2)cc1]

To a degassed solution of N-(4-(4,4,5,5-tetramethyl-1,3,2-dioxaborolan-2-yl)phenyl)acrylamide (0.34 g, 1.26 mmol) (synthesis carried out as described in reference WO/2009/056837) and tert-butyl 5-(2-(4-bromophenyl)propanamido)-3-cyclopropyl-1H-pyrazole-1-carboxylate (0.5 g, 1.15 mmol) (Inetmediate-4) in 1,4-Dioxane (10 mL) and water (2 mL) was added Cs₂CO₃ (0.74 g, 2.3 mmol). The reaction mass was allowed to stir for 10 min with degassing and added Pd(PPh₃)₄ (0.066 g, 0.057 mol), heated the reaction mass for 4 h at 100° C. in a sealed tube. The reaction mass was cooled and diluted with brine solution. The aqueous layer was separated and re-extracted with ethyl acetate. The combined organic layer was evaporated to dryness and crude material was purified by silica column chromatography by eluting with 10% methanol in DCM to get desired pure compound (0.1 g, 21%). ¹HNMR (DMSO-d₆, 400MHz): δ 12.0 (brs, 1H), 10.42 (s, 1H), 10.17 (s, 1H), 7.75 (d, 2H), 7.62-7.57 (m, 4H), 7.43 (d, 2H), 6.48-6.41 (m, 1H), 6.28 (d, 1H), 6.14 (s, 1H), 5.78 (m, 1H), 3.87-3.85 (m, 1H), 1.81-1.80 (m, 1H), 1.39 (d, 3H), 0.88-0.86 (m, 2H), 0.60-0.58 (m, 2H). LCMS: m/z=401.15 (M+H)⁺, HPLC: 96.57%, rt: 3.09 min.

Racemic N-(4′-(1-((5-cyclopropyl-1H-pyrazol-3-yl)amino)-1-oxopropan-2-yl)-[1,1′-biphenyl]-4-yl)acrylamide (0.1g, Compound-1) was separated by using chiral prep column. (Method: Column: Lux 5 μ Cellulose-4(10.0×250 mm), Elution: isocratic (95:5), A=ACN, B=0.1% DEA in EtOH) to afford the pure Isomer-1 (0.04 g) and Isomer-2 (0.04 g).

Isomer-1 (Compound-2): ¹HNMR (DMSO-d₆, 400MHz): δ 12.0 (brs, 1H), 10.42 (s, 1H), 10.17 (s, 1H), 7.75 (d, 2H), 7.62-7.57 (m, 4H), 7.43 (d, 2H), 6.48-6.41 (m, 1H), 6.28 (d, 1H), 6.14 (s, 1H), 5.78 (m, 1H), 3.87-3.85 (m, 1H), 1.81-1.80 (m, 1H), 1.39 (d, 3H), 0.88-0.86 (m, 2H), 0.60-0.58 (m, 2H). LCMS: m/z=401.15 (M+H)⁺; HPLC: 98.26%, rt: 7.01 min.; Chiral HPLC: 99.4%, rt: 7.54 min.

Isomer-2 (Compound-3): ¹H NMR (DMSO-d₆, 400MHz): δ 12.0 (brs, 1H), 10.42 (s, 1H), 10.17 (s, 1H), 7.75 (d, 2H), 7.62-7.57 (m, 4H), 7.43 (d, 2H), 6.48-6.41 (m, 1H), 6.28 (d, 1H), 6.14 (s, 1H), 5.78 (m, 1H), 3.87-3.85 (m, 1H), 1.81-1.80 (m, 1H), 1.39 (d, 3H), 0.88-0.86 (m, 2H), 0.60-0.58 (m, 2H). LCMS: m/z=401.15 (M+H)⁺; HPLC: 96.50%, rt: 7.00 min.; Chiral HPLC: 98.63%, rt: 8.58 min.

In some embodiments, one of Compound-2 and Compound-3 has an (R)-enantiomeric stereoconfiguration and the other of Compound-2 and Compound-3 has an (S)-enantiomeric stereoconfiguration. In one embodiment, Compound-2 is (R)-N-(4′-(1-((5-cyclopropyl-1H-pyrazol-3-yl)amino)-1-oxopropan-2-yl)[1,1′-biphenyl]-4-yl)acrylamide and Compound-3 is (S)-N-(4′-(1((5-cyclopropyl-1H-pyrazol-3-yl)amino)-1-oxopropan-2-yl)-[1,1′-biphenyl]-4-yl)acrylamide. In another embodiment, Compound-2 is (S)-N-(4′-(1-((5-cyclopropyl-1H-pyrazol-3-yl)amino)-1-oxopropan-2-yl)-[1,1′-biphenyl]-4-yl)acrylamide and Compound-3 is (R)-N-(4′-(1((5-cyclopropyl-1H-pyrazol-3 -yl)amino)-1-oxopropan-2-yl)-[1,1′-biphenyl]-4-yl)acrylamide.

The compounds listed in below Table-1 were prepared by a procedure similar to the one described in Example-1 with appropriate variations in reactants, quantities of reagents, protections and deprotections, solvents and reaction conditions. The characterization data of the compounds are summarized herein the below table.

TABLE-1

| Comp | | |
|---|---|---|
| No. | Structure | Characterization Data |
| 4 | 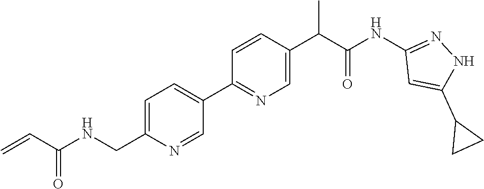 [SMILES: C=CC(=O)NCc1ccc(-c2ccc(C(C)C(=O)Nc3cc(C4CC4)[nH]n3)cn2)cn1] | ¹HNMR (DMSO-d₆, 400 MHz): δ 12.2 (s, 1H), 10.55 (s, 1H), 9.15-9.14 (d, 1H), 8.78- 8.75 (m, 1H), 8.66 (d, 1H), 8.38-8.35 (m, 1H), 7.99 (d, 1H), 7.87-7.85 (m, 1H), 7.39 (d, 1H), 6.38-6.31 (m, 1H), 6.16-6.11 (m, 2H), 5.66-5.63 (m, 1H), 4.50 (d, 2H), 3.95-3.93 (m, 1H), 1.81-1.80 (m, 1H), 1.44 (d, 3H), 0.88-0.86 (m, 2H), 0.62-0.61 (m, 2H). LCMS: m/z = 417 (M + H)⁺; HPLC: 99.70%, rt: 6.01 min. |
| 5 | 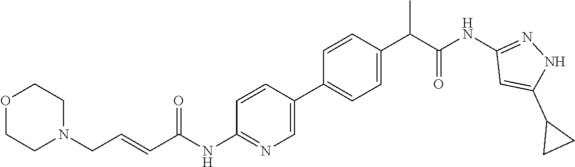 [SMILES: CC(C(=O)Nc1cc(C2CC2)[nH]n1)c1ccc(-c2ccc(NC(=O)/C=C/CN3CCOCC3)nc2)cc1] | ¹HNMR (CD₃OD, 400 MHz): δ 8.55 (d, 1H), 8.24-8.22 (m, 1H), 8.03-8.00 (m, 1H), 7.60- 7.52 (m, 2H), 7.49-7.47 (m, 2H), 6.99-6.92 (m, 1H), 6.42-6.38 (m, 1H), 6.18 (s, 1H), 3.87-3.85 (m, 1H), 3.72-3.70 (m, 4H), 3.22- 3.21 (m, 2H), 2.52-2.51 (m, 4H), 1.87-1.83 (m, 1H), 1.51 (d, 3H), 0.94-0.91 (m, 2H), 0.70-0.66 (m, 2H). LCMS: m/z = 501.10 (M + H)⁺; HPLC: 95.81%, rt: 5.89 min. |
| 6 | 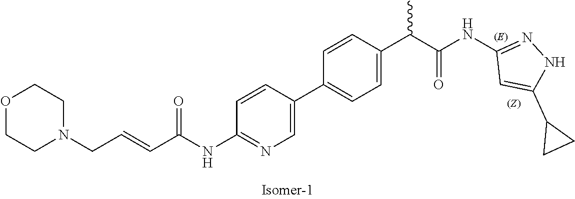 [SMILES: CC(C(=O)Nc1cc(C2CC2)[nH]n1)c1ccc(-c2ccc(NC(=O)/C=C/CN3CCOCC3)nc2)cc1] | Isomer-1 of compound 5: ¹HNMR (CD₃OD, 400 MHz): δ 8.55 (d, 1H), 8.24- 8.22 (m, 1H), 8.03-8.00 (m, 1H), 7.60-7.52 (m, 2H), 7.49-7.47 (m, 2H), 6.99-6.92 (m, 1H), 6.42-6.38 (m, 1H), 6.18 (s, 1H), 3.87- 3.85 (m, 1H), 3.72-3.70 (m, 4H), 3.22-3.21 (m, 2H), 2.52-2.51 (m, 4H), 1.87-1.83 (m, 1H), 1.51 (d, 3H), 0.94-0.91 (m, 2H), 0.70- 0.66 (m, 2H). LCMS: m/z = 501.10 (M + H)⁺; HPLC: 95.15%, rt: 5.22 min. Chiral HPLC: 99.58%, rt: 5.50 min. |
| 7 | 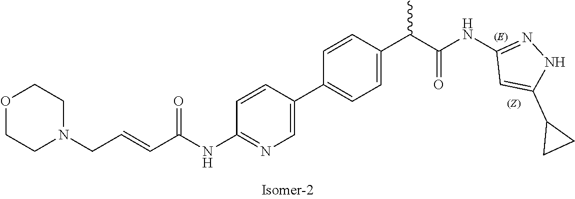 [SMILES: CC(C(=O)Nc1cc(C2CC2)[nH]n1)c1ccc(-c2ccc(NC(=O)/C=C/CN3CCOCC3)nc2)cc1] | Isomer-2 of compound 5: ¹HNMR (CD₃OD, 400 MHz): δ 8.55 (d, 1H), 8.24- 8.22 (m, 1H), 8.03-8.00 (m, 1H), 7.60-7.52 (m, 2H), 7.49-7.47 (m, 2H), 6.99-6.92 (m, 1H), 6.42-6.38 (m, 1H), 6.18 (s, 1H), 3.87- 3.85 (m, 1H), 3.72-3.70 (m, 4H), 3.22-3.21 (m, 2H), 2.52-2.51 (m, 4H), 1.87-1.83 (m, 1H), 1.51 (d, 3H), 0.94-0.91 (m, 2H), 0.70- 0.66 (m, 2H). LCMS: m/z = 501.10 (M + H)⁺; HPLC: 97.48%, rt: 5.21 min. Chiral HPLC: 99.18%, rt: 13.11 min. |
| 8 | 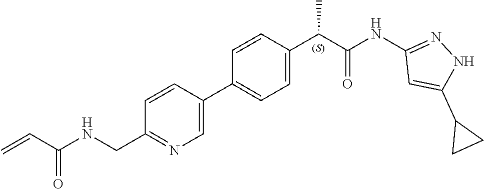 | ¹HNMR (CD₃OD, 400 MHz): δ 8.71 (s, 1H), 8.04-8.02 (m, 1H), 7.62-7.60 (m, 2H), 7.51- 7.50 (m, 2H), 7.43-7.42 (m, 1H), 6.37-6.32 (m, 1H), 6.28-6.25 (m, 1H), 6.18 (s, 1H), 5.72-5.69 (m, 1H), 4.59 (s, 2H), 3.87-3.85 (m, 1H), 1.86-1.85 (m, 1H), 1.52 (m, 3H), 0.94-0.93 (m, 2H), 0.68-0.67 (m, 2H). LCMS: m/z = 416.4 (M + H)⁺; HPLC: 99.32%, rt: 6.33 min., Chiral HPLC: 99.05%, rt: 7.58 min. |
| 29 | 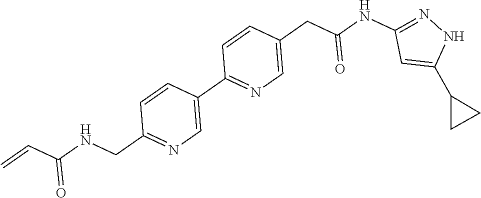 [SMILES: C=CC(=O)NCc1ccc(-c2ccc(CC(=O)Nc3cc(C4CC4)[nH]n3)cn2)cn1] | ¹HNMR (DMSO-d₆, 400 MHz): δ 12.07 (s, 1H), 10.59 (s, 1H), 9.16-9.15 (m, 1H), 8.78- 8.75 (m, 1H), 8.59 (s, 1H), 8.43-8.37 (m, 1H), 7.99-7.97 (m, 1H), 7.83-7.81 (m, 1H), 7.40-7.35 (m, 1H), 6.38-6.31 (m, 1H), 6.18- 6.13 (m, 2H), 5.66-5.63 (m, 1H), 4.51-4.39 (m, 1H), 3.68 (s, 2H), 2.22-2.17 (m, 1H), 1.85-1.79 (m, 1H), 0.89-0.87 (m, 2H), 0.64- 0.61 (m, 2H). LCMS: m/z = 403.20 (M + H)⁺; HPLC: 97.58%, rt: 5.52 min. |

### Example-2

### Synthesis of N-((4′-(1-((5-cyclopropyl-1H-pyrazol-3-yl)amino)-1-oxopropan-2-yl)-[1,1′-biphenyl]-4-yl)methyl)acrylamide (Compound-9)

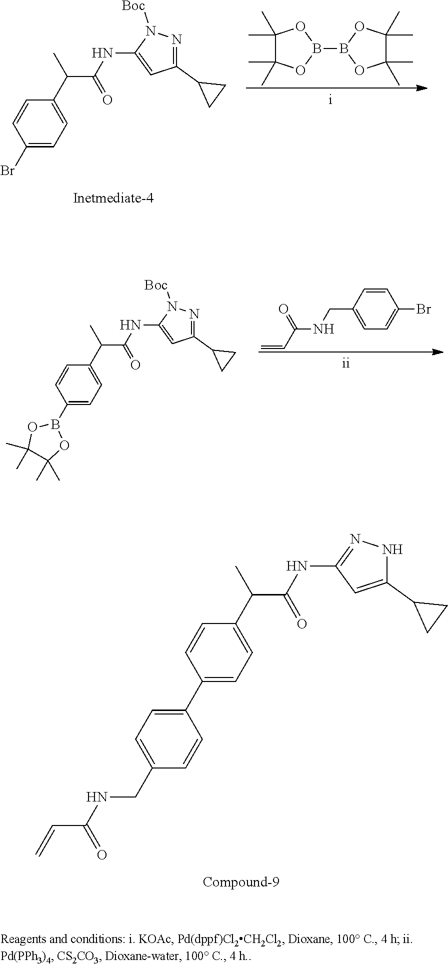

Step-i: Synthesis of tert-butyl 3-cyclopropyl-5-(2-(4-(4,4,5,5-tetramethyl-1,3,2-dioxaborolan-2-yl)phenyl)propanamido)-1H-pyrazole-1-carboxylate

To a degassed solution of tert-butyl 5-(2-(4-bromophenyl)propanamido)-3-cyclopropyl-1H-pyrazole-1-carboxylate (4 g, 9.20 mmol) (Intermediate-4) and 4,4,4′,4′,5,5,5′,5′-octamethyl-2,2′-bi(1,3,2-dioxaborolane) (2.6 g, 10.10 mol) in 1,4-Dioxane (100 mL) was added potassium acetate (1.81 g, 18.4 mmol). The reaction mass was allowed to stir for 10 min with degassing at RT and added PdCl₂(dppf)DCM complex (0.38 g, 0.46 mmol). The reaction mass was heated for 4 h at 100° C. Reaction mixture cooled to RT and filtered on celite bed, filtrate evaporated to get dark brown liquid. The crude material was purified by silica column chromatography by eluting with 20% ethyl acetate in hexane to afford title compound (3.5 g, 79%), LCMS: m/z=482.2 (M+H)⁺.

Step-ii: Synthesis of N-((4′-(1-((5-cyclopropyl-1H-pyrazol-3-yl)amino)-1-oxopropan-2-yl)-[1,1′-biphenyl]-4-yl)methyl)acrylamide

To a degassed solution of tert-butyl 3-cyclopropyl-5-(2-(4-(4,4,5,5-tetramethyl-1,3,2-dioxaborolan-2-yl)phenyl)propanamido)-1H-pyrazole-1-carboxylate (3.5 g, 7.24 mmol) and N-(4-bromobenzyl)acrylamide (1.72 g, 7.24 mmol) (synthesis carried out as described in reference WO2016/193939 A1) in 1,4-Dioxane (30 mL) and water (7 mL) was added Cs₂CO₃ (4.68 g, 14.4 mmol). The reaction mass was allowed to stir for 10 min with degassing and added Pd(PPh₃)₄ (0.42 g, 0.036 mmol), heated the reaction mass for 4 h at 100° C. in a sealed tube. Reaction mixture cooled to RT and filtered on celite bed, layers were separated for filtrate and re-extracted aqueous layer with ethyl acetate. The combined organic layer was evaporated to dryness and crude material was purified by silica column chromatography by eluting with 10% methanol in DCM to get desired pure compound (0.95 g, 33%). ¹HNMR (DMSO-d₆, 400MHz): δ 12.1 (s, 1H), 10.42 (s, 1H), 8.64-8.61 (m, 1H), 7.60-7.56 (m, 3H), 7.44-7.42 (m, 2H), 7.34-7.32 (m, 2H), 6.31-6.24 (m, 2H), 6.15-6.10 (m, 2H), 5.63-5.60 (m, 1H), 4.37 (d, 2H), 3.88-3.84 (m, 1H), 1.82-1.78 (m, 1H), 1.39 (d, 3H), 0.88-0.86 (m, 2H), 0.62-0.61 (m, 2H). LCMS: m/z=415.4 (M+H)⁺; HPLC: 90.8%, rt: 6.27 min.

Racemic N-((4′-(1-((5-cyclopropyl-1H-pyrazol-3-yl)amino)-1-oxopropan-2-yl)-[1,1′-biphenyl]-4-yl)methyl)acrylamide (0.95 g, Compound-9) was separated by using chiral prep column. (Method: CELLULOSE-4 (250mm×10 mm), 5.00, Elution: isocratic (70:30), A=Hexane, B=0.1% DEA in Et0H) to afford the pure Isomer-1 (0.35 g) and Isomer-2 (0.34 g).

Isomer-1 (Compound-10): ¹HNMR (DMSO-d₆, 400MHz): δ 12.1 (s, 1H), 10.42 (s, 1H), 8.64-8.61 (m, 1H), 7.60-7.56 (m, 3H), 7.44-7.42 (m, 2H), 7.34-7.32 (m, 2H), 6.31-6.24 (m, 2H), 6.15-6.10 (m, 2H), 5.63-5.60 (m, 1H), 4.37 (d, 2H), 3.88-3.84 (m, 1H), 1.82-1.78 (m, 1H), 1.39 (d, 3H), 0.88-0.86 (m, 2H), 0.62-0.61 (m, 2H). LCMS: m/z=415.4 (M+H)⁺; HPLC: 91.01%, rt: 11.43 min.; Chiral HPLC: 92.54%, rt: 8.03 min.

Isomer-2 (Compound-11): ¹HNMR (DMSO-d₆, 400MHz): δ 12.1 (s, 1H), 10.42 (s, 1H), 8.64-8.61 (m, 1H), 7.60-7.56 (m, 3H), 7.44-7.42 (m, 2H), 7.34-7.32 (m, 2H), 6.31-6.24 (m, 2H), 6.15-6.10 (m, 2H), 5.63-5.60 (m, 1H), 4.37 (d, 2H), 3.88-3.84 (m, 1H), 1.82-1.78 (m, 1H), 1.39 (d, 3H), 0.88-0.86 (m, 2H), 0.62-0.61 (m, 2H). LCMS: m/z=415.4 (M+H)⁺; HPLC: 98.38%, rt: 5.02 min.; Chiral HPLC: 96.45%, rt: 6.65 min.

In some embodiments, one of Compound 6 and Compound 7 has an (R)-enantiomeric stereoconfiguration and the other of Compound 6 and Compound 7 has an (S)-enantiomeric stereoconfiguration. In one embodiment, Compound 6 is (R,E)-N-(5-(4-(1((5-cyclopropyl-1H-pyrazol-3-yl)amino)-1-oxopropan-2-yl)phenyl)pyridin-2-yl)-4-morpholinobut-2-enamide and Compound 7 is (S,E)-N-(5-(4-(1-((5-cyclopropyl-1H-pyrazol-3-yl)amino)-1-oxopropan-2-yl)phenyl)pyridin-2-yl)-4-morpholinobut-2-enamide. In another embodiment, Compound 6 is (S,E)-N-(5-(4-(1-((5-cyclopropyl-1H-pyrazol-3 -yl)amino)-1-oxopropan-2-yl)phenyl)pyridin-2-yl)-4-morpholinobut-2-enamide and Compound 7 is (R,E)-N-(5-(4-(1-((5-cyclopropyl-1H-pyrazol-3-yl)amino)-1-oxopropan-2-yl)phenyl)pyridin-2-yl)-4-morpholinobut-2-enamide.

In some embodiments, one of Compound 10 and Compound 11 has an (R)-enantiomeric stereoconfiguration and the other of Compound 10 and Compound 11 has an (S)-enantiomeric stereoconfiguration. In one embodiment, Compound 10 is (R)-N-((4′-(1-((5-cyclopropyl-1H-pyrazol-3 -yl)amino)-1-oxopropan-2-yl)- [1,1′-biphenyl]-4-yl)methyl)acrylamide and Compound 11 is (S)-N-((4′-(1-((5-cyclopropyl-1H-pyrazol-3-yl)amino)-1-oxopropan-2-yl)-[1,1′-biphenyl]-4-yl)methyl)acrylamide. In another embodiment, Compound 10 is (S)-N-((4′-(1-((5-cyclopropyl-1H-pyrazol-3 -yl)amino)-1-oxopropan-2-yl)-[1,1′-biphenyl]-4-yl)methyl)acrylamide and Compound 11 is (R)-N-((4′-(1-((5 -cyclopropyl-1H-pyrazol-3-yl)amino)-1-oxopropan-2-yl)- [1,1′-biphenyl]-4-yl)methyl)acrylamide.

The compounds listed in below Table-2 were prepared by a procedure similar to the one described in Example-2 with appropriate variations in reactants, quantities of reagents, protections and deprotections, solvents and reaction conditions. The characterization data of the compounds are summarized herein the below table.

TABLE-2

| Comp | | |
|---|---|---|
| No. | Structure | Characterization Data |
| 12 | 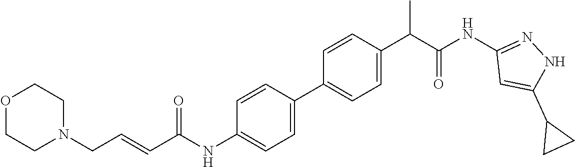 [SMILES: CC(C(=O)Nc1cc(C2CC2)[nH]n1)c1ccc(-c2ccc(NC(=O)/C=C/CN3CCOCC3)cc2)cc1] | ¹HNMR (CD₃OD, 400 MHz): δ 7.69-7.67 (m, 2H), 7.59-7.55 (m, 4H), 7.43 (d, 2H), 6.93-6.86 (m, 1H), 6.34-6.30 (m, 1H), 6.16 (s, 1H), 3.87-3.85 (m, 1H), 3.72- 3.70 (m, 4H), 3.22-3.21 (m, 2H), 2.58-2.51 (m, 4H), 1.88-1.81 (m, 1H), 1.52 (d, 3H), 0.94-0.93 (m, 2H), 0.70-0.68 (m, 2H). LCMS: m/z = 500.10 (M + H)⁺; HPLC: 95.44%, rt: 5.35 min. |
| 13 | 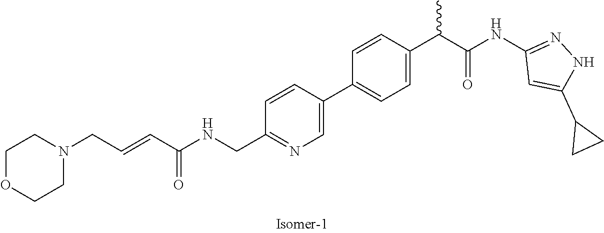 [SMILES: CC(C(=O)Nc1cc(C2CC2)[nH]n1)c1ccc(-c2ccc(CNC(=O)/C=C/CN3CCOCC3)nc2)cc1] | Isomer-1: ¹HNMR (CD₃OD, 400 MHz): δ 8.71 (d, 1H), 8.04-8.01 (m, 1H), 7.62-7.60 (m, 2H), 7.51- 7.49 (m, 2H), 7.43-7.41 (m, 1H), 6.83-6.78 (m, 1H), 6.24-6.19 (m, 1H), 6.15 (s, 1H), 4.58 (s, 2H), 3.88-3.86 (m, 1H), 3.71-3.67 (m, 4H), 3.18-3.15 (m, 2H), 2.50-2.47 (m, 4H), 1.88-1.81 (m, 1H), 1.52 (d, 3H), 0.94-0.91 (m, 2H), 0.68- 0.66 (m, 2H). LCMS: m/z = 515.10 (M + H)⁺; HPLC: 97.91%, rt: 4.98 min. Chiral HPLC: 97.64, rt: 5.0 min. |
| 14 | 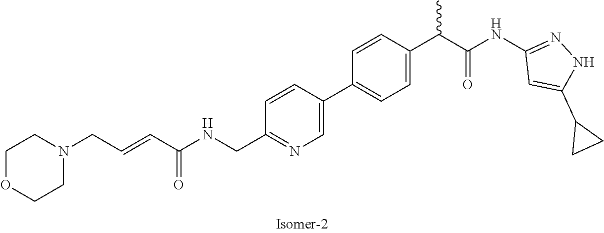 [SMILES: CC(C(=O)Nc1cc(C2CC2)[nH]n1)c1ccc(-c2ccc(CNC(=O)/C=C/CN3CCOCC3)nc2)cc1] | Isomer-2: ¹HNMR (CD₃OD, 400 MHz): δ 8.71 (d, 1H), 8.04-8.01 (m, 1H), 7.62-7.60 (m, 2H), 7.51- 7.49 (m, 2H), 7.43-7.41 (m, 1H), 6.83-6.78 (m, 1H), 6.24-6.19 (m, 1H), 6.15 (s, 1H), 4.58 (s, 2H), 3.88-3.86 (m, 1H), 3.71-3.67 (m, 4H), 3.18-3.15 (m, 2H), 2.50- 2.47 (m, 4H), 1.88-1.81 (m, 1H), 1.52 (d, 3H), 0.94-0.91 (m, 2H), 0.68-0.66 (m, 2H). LCMS: m/z = 515.10 (M + H)⁺; HPLC: 97.82%, rt: 4.99 min. Chiral HPLC: 95.10, rt: 7.39 min. |
| 15 | 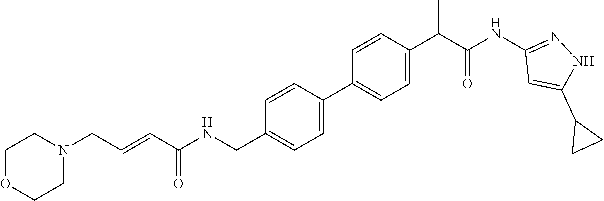 [SMILES: CC(C(=O)Nc1cc(C2CC2)[nH]n1)c1ccc(-c2ccc(CNC(=O)/C=C/CN3CCOCC3)cc2)cc1] | ¹HNMR (CD₃OD, 400 MHz): 7.57- 7.55 (m, 4H), 7.45-7.43 (m, 2H), 7.35-7.33 (m, 2H), 6.85-6.78 (m, 1H), 6.18-6.10 (m, 2H), 4.45 (s, 2H), 3.85-3.80 (m, 1H), 3.71-3.68 (m, 4H), 3.16-3.14 (m, 2H), 2.50- 2.47 (m, 4H), 1.88-1.81 (m, 1H), 1.52 (d, 3H), 0.94-0.91 (m, 2H), 0.68-0.66 (m, 2H). LCMS: m/z = 514.15 (M + H)⁺; HPLC: 96.73%, rt: 7.40 min. |
| 16 | 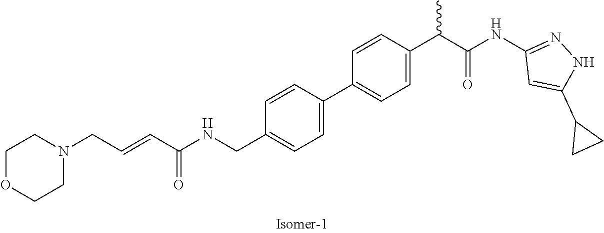 [SMILES: CC(C(=O)Nc1cc(C2CC2)[nH]n1)c1ccc(-c2ccc(CNC(=O)/C=C/CN3CCOCC3)cc2)cc1] | Isomer-1 of compound 15: ¹HNMR (CD₃OD, 400 MHz): 7.57- 7.55 (m, 4H), 7.45-7.43 (m, 2H), 7.35-7.33 (m, 2H), 6.85-6.78 (m, 1H), 6.18-6.10 (m, 2H), 4.45 (s, 2H), 3.85-3.80 (m, 1H), 3.71-3.68 (m, 4H), 3.16-3.14 (m, 2H), 2.50- 2.47 (m, 4H), 1.88-1.81 (m, 1H), 1.52 (d, 3H), 0.94-0.91 (m, 2H), 0.68-0.66 (m, 2H). LCMS: m/z = 514.15 (M + H)⁺; HPLC: 98.28%, rt: 6.10 min. Chiral HPLC: 94.70, rt: 4.55 min. |
| 17 | 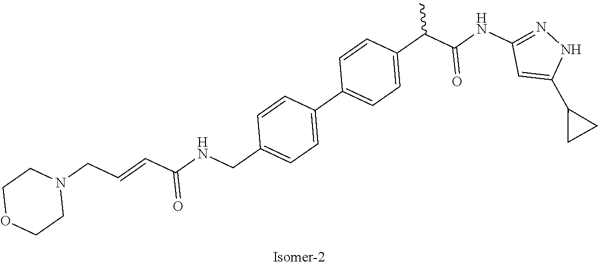 [SMILES: CC(C(=O)Nc1cc(C2CC2)[nH]n1)c1ccc(-c2ccc(CNC(=O)/C=C/CN3CCOCC3)cc2)cc1] | Isomer-2 of compound 15: ¹HNMR (CD₃OD, 400 MHz): 7.57- 7.55 (m, 4H), 7.45-7.43 (m, 2H), 7.35-7.33 (m, 2H), 6.85-6.78 (m, 1H), 6.18-6.10 (m, 2H), 4.45 (s, 2H), 3.85-3.80 (m, 1H), 3.71- 3.68 (m, 4H), 3.16-3.14 (m, 2H), 2.50-2.47 (m, 4H), 1.88-1.81 (m, 1H), 1.52 (d, 3H), 0.94-0.91 (m, 2H), 0.68-0.66 (m, 2H). LCMS: m/z = 514.15 (M + H)⁺; HPLC: 97.85%, rt: 6.10 min. Chiral HPLC: 89.09, rt: 6.41 min. |
| 18 | 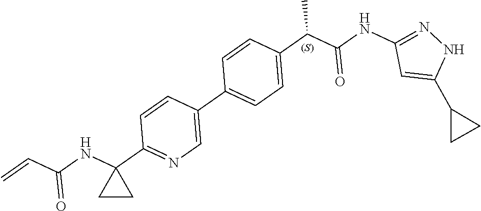 | ¹HNMR (DMSO-d₆, 400 MHz): δ 12.2 (s, 1H), 10.43 (s, 1H), 8.95 (s, 1H), 8.71 (d, 1H), 7.94-7.92 (m, 1H), 7.62-7.60 (m, 2H), 7.46- 7.44 (m, 2H), 7.30 (d, 1H), 6.36- 6.29 (m, 1H), 6.16-6.11 (m, 2H), 5.67-5.64 (m, 1H), 3.89-3.87 (m, 1H), 1.81-1.80 (m, 1H), 1.53-1.50 (m, 2H), 1.40 (d, 3H), 1.18-1.16 (m, 2H), 0.88-0.86 (m, 2H), 0.62- 0.61 (m, 2H). LCMS: m/z = 442.60 (M + H)⁺; HPLC: 99.73%, rt: 6.50 min. Chiral HPLC: 92.48, rt: 9.45 min. |
| 19 | 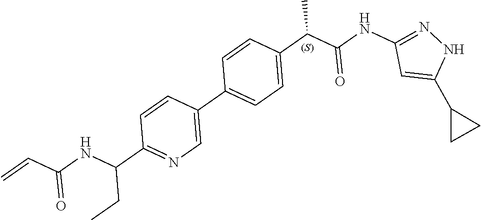 | ¹HNMR (CDCl3, 400 MHz): δ 8.68 (m, 1H), 8.36 (d, 1H), 7.77-7.74 (m, 1H), 7.47-7.46 (m, 2H), 7.40-7.37 (m, 2H), 7.28-7.26 (m, 1H), 7.09-7.08 (m, 1H), 6.33-6.29 (m, 2H), 6.23-6.18 (m, 1H), 5.65-5.62 (m, 1H), 5.13-5.08 (m, 1H), 3.75-3.70 (m, 1H), 1.95-1.89 (m, 3H), 1.87-1.75 (m, 1H), 1.58 (d, 3H), 0.94-0.88 (m, 2H), 0.71-0.67 (m, 2H). LCMS: m/z = 444.65 (M + H)⁺; HPLC: 94.31%, rt: 6.50 min. |
| 20 | 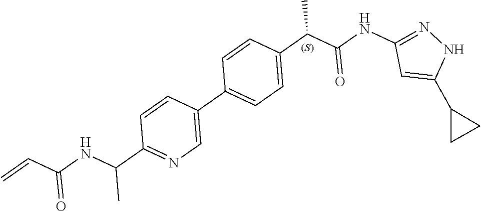 | ¹HNMR (CDCl3, 400 MHz): δ 8.69 (s, 1H), 7.83-7.80 (m, 2H), 7.51-7.49 (m, 2H), 7.41-7.40 (m, 2H), 7.31-7.30 (m, 1H), 7.06-7.05 (m, 1H), 6.33-6.29 (m, 2H), 6.21-6.16 (m, 1H), 5.65-5.62 (m, 1H), 5.26-5.25 (m, 1H), 3.73-3.71 (m, 1H), 1.77-1.70 (m, 1H), 1.60-1.59 (m, 3H), 1.53 (d, 3H), 0.93-0.91 (m, 2H), 0.70-0.68 (m, 2H). LCMS: m/z = 430.1 (M + H)⁺; HPLC: 96.66%, rt: 5.64 min. |
| 21 | 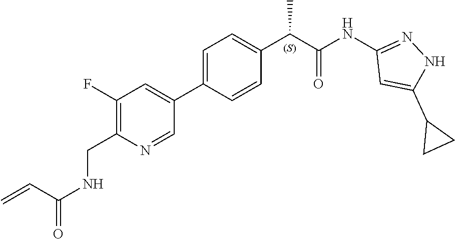 | ¹HNMR (DMSO-d₆, 400 MHz): δ 12.0 (s, 1H), 10.46 (s, 1H), 8.72-8.68 (m, 2H), 8.10 (d, 1H), 7.72-7.70 (m, 2H), 7.49-7.47 (m, 2H), 6.38-6.30 (m, 1H), 6.12-6.10 (m, 2H), 5.61-5.60 (m, 1H), 4.55-4.53 (m, 2H), 3.81-3.78 (m, 1H), 1.83-1.80 (m, 1H), 1.39 (d, 3H), 0.88-0.86 (m, 2H), 0.62- 0.61 (m, 2H). LCMS: m/z = 434.0 (M + H)⁺; HPLC: 98.38%, rt: 6.71 min.; Chiral HPLC: 94.63%, rt: 8.78 min. |
| 22 | 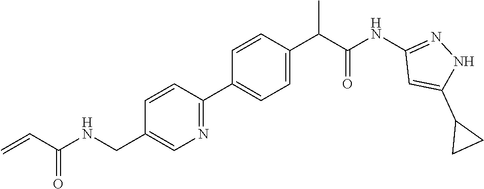 [SMILES: C=CC(=O)NCc1ccc(-c2ccc(C(C)C(=O)Nc3cc(C4CC4)[nH]n3)cc2)nc1] | ¹HNMR (DMSO-d₆, 400 MHz): δ 12.0 (s, 1H), 10.46 (s, 1H),8.72- 8.68 (m, 2H), 8.10 (d, 1H), 7.72-7.70 (m, 2H), 7.49-7.47 (m, 2H), 6.38-6.30 (m, 1H), 6.12-6.10 (m, 2H), 5.61-5.60 (m, 2H), 4.55-4.53 (m, 2H), 3.81- 3.78 (m, 1H), 1.83-1.80 (m, 1H), 1.39 (d, 3H), 0.88-0.86 (m, 2H), 0.62-0.61 (m, 2H). LCMS: m/z = 416.1 (M + H)⁺; HPLC: 99.35%, rt: 6.16 min.; Chiral HPLC: 90.02%, rt: 15.37 min. |

In some embodiments, provided herein is the (R)-enantiomer of compound 12, i.e., (R,E)-N-(4′-(1-((5-cyclopropyl-1H-pyrazol-3-yl)amino)-1-oxopropan-2-yl)-[1,1′-biphenyl]-4-yl)-4-morpholinobut-2-enamide. In other embodiments, provided herein is the (S)-enantiomer of compound 12, i.e., (S,E)-N-(4′-(1-((5-cyclopropyl-1H-pyrazol-3-yl)amino)-1-oxopropan-2-yl)- [1,1′-biphenyl]-4-yl)-4-morpholinobut-2-enamide.

In some embodiments, compounds 13 & 14 were isolated from the chiral separation of their racemic compound.

In some embodiments, one of Compound 13 and Compound 14 has an (R)-enantiomeric stereoconfiguration and the other of Compound 13 and Compound 14 has an (S)-enantiomeric stereoconfiguration. In one embodiment, Compound 13 is (R,E)-N-((5-(4-(1-((5-cyclopropyl-1H-pyrazol-3-yl)amino)-1-oxopropan-2-yl)phenyl)pyridin-2-yl)methyl)-4-morpholinobut-2-enamide and Compound 14 is (S,E)-N-((5-(4-(1-((5-cyclopropyl-1H-pyrazol-3 -yl)amino)-1-oxopropan-2-yl)phenyl)pyridin-2-yl)methyl)-4-morpholinobut-2-enamide. In another embodiment, Compound 13 is (S,E)-N-((5-(4-(1-((5-cyclopropyl-1H-pyrazol-3-yl)amino)-1-oxopropan-2-yl)phenyl)pyridin-2-yl)methyl)-4-morpholinobut-2-enamide and Compound 14 is (R,E)-N-((5-(4-(1-((5-cyclopropyl-1H-pyrazol-3-yl)amino)-1-oxopropan-2-yl)phenyl)pyridin-2-yl)methyl)-4-morpholinobut-2-enamide.

In some embodiments, one of Compound 16 and Compound 17 has an (R)-enantiomeric stereoconfiguration and the other of Compound 16 and Compound 17 has an (S)-enantiomeric stereoconfiguration. In one embodiment, Compound 16 is (R,E)-N-((4′-(1-((5-cyclopropyl-1H-pyrazol-3 -yl)amino)-1-oxopropan-2-yl)- [1,1′-biphenyl]-4-yl)methyl)-4-morpholinobut-2-enamide and Compound 17 is (S,E)-N-((4′-(1-((5-cyclopropyl-1H-pyrazol-3 -yl)amino)-1-oxopropan-2-yl)-[1,1′-biphenyl]-4-yl)methyl)-4-morpholinobut-2-enamide. In another embodiment, Compound 16 is (S,E)-N-((4′-(1-((5-cyclopropyl-1H-pyrazol-3-yl)amino)-1-oxopropan-2-yl)-[1,1′-biphenyl]-4-yl)methyl)-4-morpholinobut-2-enamide and Compound 17 is (R,E)-N-((4′-(1-((5-cyclopropyl-1H-pyrazol-3 -yl)amino)-1-oxopropan-2-yl)-[1,1′-biphenyl]-4-yl)methyl)-4-morpholinobut-2-enamide

In some embodiments, provided herein is the (R)-enantiomer of compound 22, i.e., (R)-N-((6-(4-(1-((5-cyclopropyl-1H-pyrazol-3 -yl)amino)-1-oxopropan-2-yl)phenyl)pyridin-3-yl)methyl)acrylamide. In other embodiments, provided herein is the (S)-enantiomer of compound 22, i.e., (S)-N-((6-(4-(1-((5-cyclopropyl-1H-pyrazol-3-yl)amino)-1-oxopropan-2-yl)phenyl)pyridin-3-yl)methyl)acrylamide.

### Example-3

### Synthesis of N-(4-(1-((5-cyclopropyl-1H-pyrazol-3-yl)amino)-1-oxopropan-2-yl)benzyl)acrylamide (Compound-23)

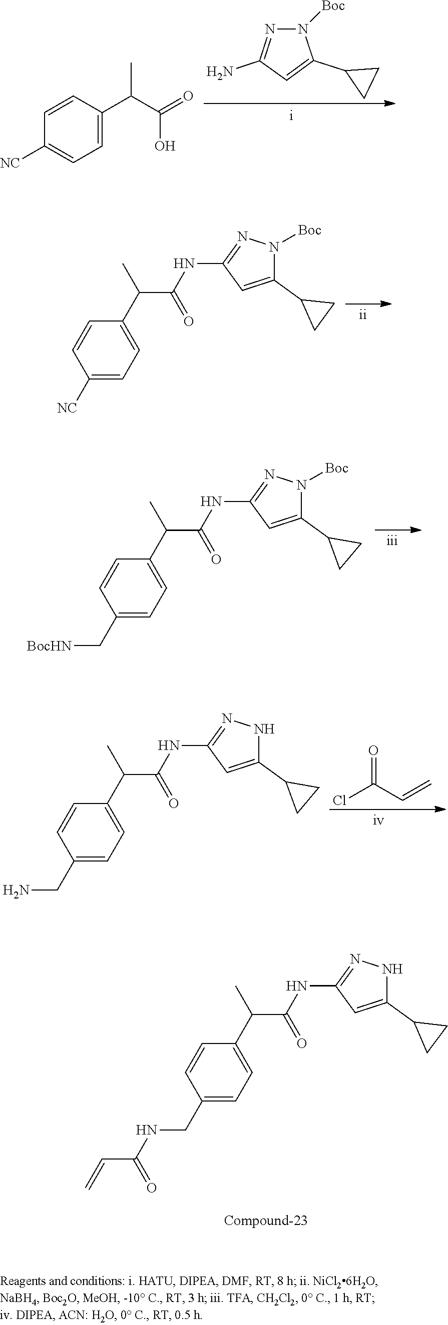

Step-i: Synthesis of tert-butyl 3-(2-(4-cyanophenyl)propanamido)-5-cyclopropyl-1H-pyrazole-1-carboxylate

To a cooled solution of 2-(4-cyanophenyl)propanoic acid (0.3 g, 1.70 mmol) in DMF (2 mL) at 0° C. was added HATU (0.97 g, 2.55 mmol) followed by DIPEA (0.43 mL, 3.4 mmol) and finally added tert-butyl 3-amino-5-cyclopropyl-1H-pyrazole-1-carboxylate (0.38 g, 1.70 mmol). The reaction mixture was stirred for 8 h at RT. The reaction mixture was quenched with ice-water and diluted with ethyl acetate. The aqueous layer was separated and extracted with ethyl acetate (2×10 mL). The combined organic phase was washed with brine, dried over Na₂SO₄, filtered and concentrated the crude residue which was purified by combi column using 20% EtOAc in hexane as a mobile phase to get 0.40 g (61%) of desired product. LCMS: m/z=381 (M+H)⁺.

Step-ii: Synthesis of tert-butyl 3-(2-(4-(((tert-butoxycarbonyl)amino)methyl)phenyl) propanamido)-5-cyclopropyl-1H-pyrazole-1-carboxylate

To a stirred suspension of tert-butyl 3-(2-(4-cyanophenyl)propanamido)-5-cyclopropyl-1H-pyrazole-1-carboxylate (0.75 g, 1.96 mmol), (Boc)₂O (0.47 g, 2.15 mmol) and NiCl₂.6H₂O (0.232 g, 0.98 mmol) in MeOH (15 mL) was added NaBH₄ (0.37 g, 9.8 mmol) at −10° C. and stirred for further 3 h at RT. After completion of reaction distilled out the solvent and diluted with water. The aqueous layer was extracted with EtOAc (25 mL×3). The combined organic extractions were washed with water, brine solution and dried over anhydrous Na₂SO₄. The organic layer was concentrated under reduced pressure and residue obtained was purified by combi column using 20%-50% EtOAc in hexane as a mobile phase to get 0.35 g (36.66%) of title product. LCMS: m/z=485.1 (M+H)⁺.

Step-iii: Synthesis of 2-(4-(aminomethyl)phenyl)-N-(5-cyclopropyl-1H-pyrazol-3-yl) propanamide

To a solution of tert-butyl 3-(2-(4-(((tert-butoxycarbonyl)amino)methyl) phenyl)propanamido)-5-cyclopropyl-1H-pyrazole-1-carboxylate (0.35 g, 1.30 mmol) in DCM (2 mL) was added TFA (1 mL) at RT and stirred at the same temperature for further 1 h under argon atmosphere. After completion of reaction distilled out the solvent and diluted the reaction mixture with water (30 mL) and then further it was basified with saturated K₂CO₃ solution (pH=˜12). The aqueous layer was extracted with 20% methanol in DCM (30 mL×3). The combined organic layer were washed with water, brine solution and dried over anhydrous Na₂SO₄ and concentrated under reduced pressure to get 0.22 g (79%) of 2-(4-(aminomethyl)phenyl)-N-(5-cyclopropyl-1H-pyrazol-3-yl)propanamide. LCMS: m/z=285.1 (M+H)⁺.

Step-iv: Synthesis of N-(4-(1-((5-cyclopropyl-1H-pyrazol-3-yl)amino)-1-oxopropan-2-yl) benzyl)acrylamide

To a solution of 2-(4-(aminomethyl)phenyl)-N-(5-cyclopropyl-1H-pyrazol-3-yl) propanamide (0.22 g, 0.57 mmol) in ACN (10 mL) was added water (2 mL), DIPEA (0.15 mL, 1.13 mmol) and acryloyl chloride (0.051 g, 0.57 mmol) at 0° C. After 30 min, the reaction mixture was quenched with ice-water and diluted with DCM. The aqueous layer was separated and extracted with DCM (2×10 mL). The combined organic phase was washed with brine, dried over Na₂SO₄, filtered and concentrated. The crude residue was purified by silica column chromatography by eluting with 0-5% MeOH-DCM to afford the title compound (0.06 g, 31%). ¹HNMR (DMSO-d₆, 400MHz): δ 12.0 (s, 1H), 10.35 (s, 1H), 8.54-8.52 (m, 1H), 7.31-7.29 (m, 2H), 7.20-7.18 (m, 2H), 6.28-6.21 (m, 1H), 6.12-6.09 (m, 2H), 5.61-5.71 (m, 1H), 4.29 (d, 2H), 3.81-3.79 (m, 1H), 1.82-1.80 (m, 1H), 1.34 (d, 3H), 0.88-0.81 (m, 2H), 0.62-0.60 (m, 2H). LCMS: m/z=339.3 (M+H)⁺; HPLC: 98.90%, rt: 6.23 min.

Racemic N-(4-(1-((5-cyclopropyl-1H-pyrazol-3-yl)amino)-1-oxopropan-2-yl)benzyl) acrylamide (0.06 g, Compound-23) was separated by using chiral prep column. (Method: CELLULOSE-4 (250 mm×10 mm), 5.00, Elution: isocratic (70:30), A=Hexane, B=0.1% TFA in EtOH) to afford the pure Isomer-1 (0.02 g) and Isomer-2 (0.02 g).

Isomer-1 (Compound-24): ¹HNMR (DMSO-d₆, 400MHz): δ 12.0 (s, 1H), 10.35 (s, 1H), 8.54-8.52 (m, 1H), 7.31-7.29 (m, 2H), 7.20-7.18 (m, 2H), 6.28-6.21 (m, 1H), 6.12-6.09 (m, 2H), 5.61-5.71 (m, 1H), 4.29 (d, 2H), 3.81-3.79 (m, 1H), 1.82-1.80 (m, 1H), 1.34 (d, 3H), 0.88-0.81 (m, 2H), 0.62-0.60 (m, 2H). LCMS: m/z=339.3 (M+H)⁺; HPLC: 97.74%, rt: 3.55 min. Chiral HPLC: 99.14%, rt: 7.63 min.

Isomer-2 (Compound-25): ¹NMR (DMSO-d₆, 400MHz): δ 12.0 (s, 1H), 10.35 (s, 1H), 8.54-8.52 (m, 1H), 7.31-7.29 (m, 2H), 7.20-7.18 (m, 2H), 6.28-6.21 (m, 1H), 6.12-6.09 (m, 2H), 5.61-5.71 (m, 1H), 4.29 (d, 2H), 3.81-3.79 (m, 1H), 1.82-1.80 (m, 1H), 1.34 (d, 3H), 0.88-0.81 (m, 2H), 0.62-0.60 (m, 2H). LCMS: m/z=339.3 (M+H)⁺; HPLC: 97.29%, rt: 3.54 min; Chiral HPLC: 94.23%, rt: 9.21 min.

In some embodiments, one of Compound 24 and Compound 25 has an (R)-enantiomeric stereoconfiguration and the other of Compound 24 and Compound 25 has an (S)-enantiomeric stereoconfiguration. In one embodiment, Compound 24 is (R)-N-(4-(1-((5-cyclopropyl-1H-pyrazol-3-yl)amino)-1-oxopropan-2-yl)benzyl)acrylamide and Compound 25 is (S)-N-(4-(1-((5-cyclopropyl-1H-pyrazol-3-yl)amino)-1-oxopropan-2-yl)benzyl) acrylamide. In another embodiment, Compound 24 is (S)-N-(4-(1-((5-cyclopropyl-1H-pyrazol-3-yl)amino)-1-oxopropan-2-yl)benzyl)acrylamide and Compound 25 is (R)-N-(4-(1-((5-cyclopropyl-1H-pyrazol-3-yl)amino)-1-oxopropan-2-yl)benzyl)acrylamide.

### Example-4

### Synthesis of N-((5-(1-((5-cyclopropyl-1H-pyrazol-3-yl)amino)-1-oxopropan-2-yl)pyridin-2-yl)methyl)acrylamide (Compound-26)

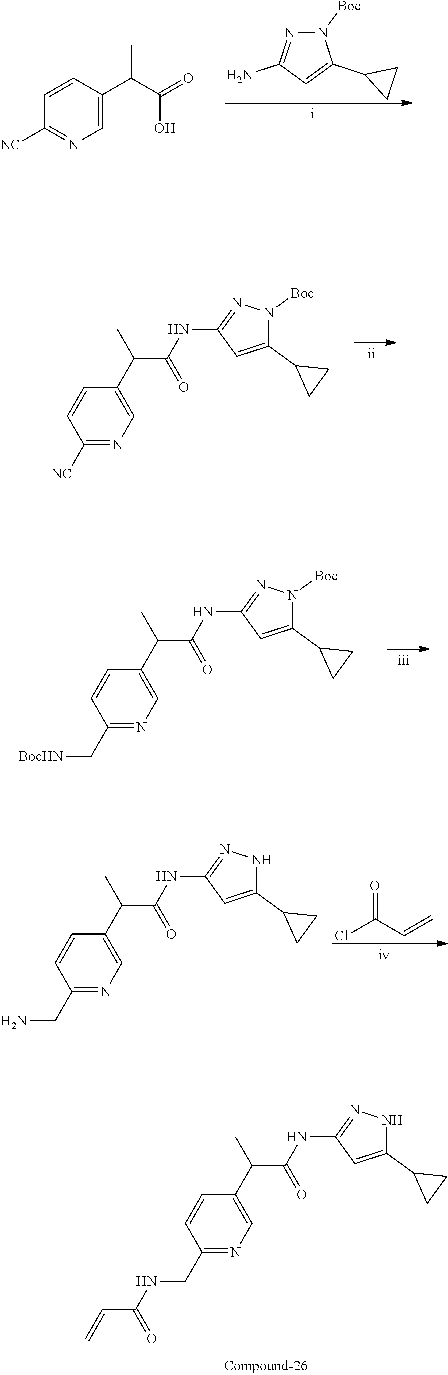

The compound-26 was prepared by a procedure similar to the one described in Example-3 by using 2-(6-cyanopyridin-3-yl)propanoic acid as starting material. ¹HNMR (DMSO-d₆, 400MHz): δ 12.0 (s, 1H), 10.48 (s, 1H), 8.67-8.65 (m, 1H), 8.47 (d, 1H), 7.72-7.70 (m, 1H), 7.24-7.22 (m, 1H), 6.33-6.26 (m, 1H), 6.12-6.09 (m, 2H), 5.63-5.59 (m, 1H), 4.39 (d, 2H), 3.87-3.85 (m, 1H), 1.80-1.79 (m, 1H), 1.38 (d, 3H), 0.88-0.83 (m, 2H), 0.62-0.60 (m, 2H). LCMS: m/z=340.05 (M+H)⁺; HPLC: 99.20%, rt: 4.95 min;

In some embodiments, provided herein is the (R)-enantiomer of compound 26, i.e., (R)-N-(5-(1-((5-cyclopropyl-1H-pyrazol-3-yl)amino)-1-oxopropan-2-yl)pyridin-2-yl)methyl)acrylamide. In other embodiments, provided herein is the (S)-enantiomer of compound 26, i.e., (S)-N4(5-(1-((5-cyclopropyl-1H-pyrazol-3-yl)amino)-1-oxopropan-2-yl)pyridin-2-yl)methyl)acrylamide.

Although the present application has been illustrated by certain of the preceding examples, it is not to be construed as being limited thereby; but rather, the present application encompasses the generic area as hereinbefore disclosed. For example, the compounds in the below table-3 can be prepared by following similar procedure as described in above Schemes/Examples with suitable modifications known to the one ordinary skilled in the art and are included in the scope of the present invention:

TABLE 3

| Comp | |
|---|---|
| No. | Structure |
| 27 | 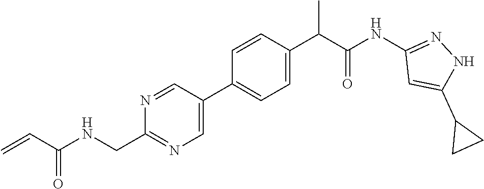 [SMILES: C=CC(=O)NCc1ncc(-c2ccc(C(C)C(=O)Nc3cc(C4CC4)[nH]n3)cc2)cn1] |
| 28 | 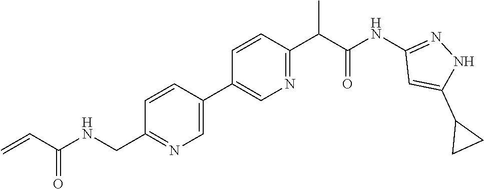 [SMILES: C=CC(=O)NCc1ccc(-c2ccc(C(C)C(=O)Nc3cc(C4CC4)[nH]n3)nc2)cn1] |

In some embodiments, provided herein is the (R)-enantiomer of compound 27 or 28. In other embodiments, provided herein is the (S)-enantiomer of compound 27 or 28.

CDK12 Plate based Target Engagement Assay:

Jurkat cells were treated with varying concentrations of the compound for 6 hours. The DMSO concentration was maintained at 0.1%. Cells were harvested and lysed. 200 μg of the lysate was incubated with 1 μM Bio-THZ531 in the presence of 1mM DTT and incubated on a rocker at 4° C. overnight. 100 μL of this sample was added to pre-washed streptavidin coated plates and incubated at room temperature on a rocker for 2 hours. The plates were washed and incubated with CDK12 antibody for overnight at 4° C. Next day the plate was washed and incubated for 2 hours with HRP labelled anti rabbit secondary antibody. Bio-THZ531 bound CDK12 was determined using TMB substrate. The plates were read using the M3 spectrophotometer at 450 nM and 570 nM. Percentage CDK12 occupancy with the test compound was calculated over untreated control. Occupancy50 was calculated by fitting the dose response data to sigmoidal curve fitting equation using GraphPad Prism software V5.

### Jurkat Cell Proliferation Assay:

Jurkat cells were seeded in a 96-well round-bottom plate and treated with varying concentration of compound. The final DMSO concentration was maintained at 0.1%. Compounds were screened in a 9-point dose response format starting with 10 μM and ⅓^{rd} serial dilution. At the end of 72 h, cells were spun down and media was aspirated. 50 μL of XTT containing media was added to the wells. The plates were read using the M3 spectrophotometer at 465 nM. EC₅₀ was calculated by fitting the dose response data to sigmoidal curve fitting equation using GraphPad Prism software V5.

Biochemical assay for CDK12:

Kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM

DTT) to remove unbound ligand and to reduce non-specific phage binding. Binding reactions were assembled by combining kinases, liganded affinity beads, and test compounds in lx binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.02 ml. The assay plates were incubated at room temperature with shaking for 1 hour and the affinity beads were washed with wash buffer (1 PBS, 0.05% Tween 20). The beads were then re-suspended in elution buffer (1× PBS, 0.05% Tween 20, 0.5 μM non-biotinylated affinity ligand) and incubated at room temperature with shaking for 30 minutes. The kinase concentration in the eluates was measured by qPCR.

Biochemical assay for CDK13:

LanthaScreen Eu Kinase Binding Assays are based on the binding and displacement of Alexa Fluor 647-labeled, ATP-competitive kinase inhibitor scaffold (kinase tracer) to the kinase of interest. Binding of the kinase tracer 236 (100 nM) to the CDK13 (5 nM) kinase is detected using a europium-labelled anti- GST tag antibody (2 nM), which binds to the kinase. Simultaneous binding of the tracer and antibody to the kinase results in a high degree of FRET (fluorescence resonance energy transfer) from the europium (Eu) donor fluorophore to the Alexa Fluor™ 647 acceptor fluorophore on the kinase tracer. Binding of an inhibitor to the kinase competes for binding with the tracer, resulting in a loss of FRET signal.

Biochemical assay for CDK7:

The inhibitory activity of the test compounds was assessed by the LANCE TR-FRET assay, which detects the ATP-dependent phosphorylation of an ULight-myelin basic protein (MBP) substrate peptide (100 nM) by CDK7 (10 nM). Briefly, the enzyme reaction was run in reaction buffer (20 mM HEPES (pH 7.5), 10 mM MgCl2, 0.01% Triton x, 100 μM Sodium Orthovanadate, 1 mM DTT). The assay was performed in 384-well plate. The end concentration of the ATP substrate was 1 mM/100 μM, and that of the ULight-MBP substrate peptide was 100 nM, and of CDK7 was 10 nM. Pre-incubation of the compound and enzyme was performed for 60 min at room temperature. After 60 min incubation at room temperature, the reaction was terminated by the addition of 40 mM EDTA and 1 nM Eu-labeled anti-phospho-MBP-binding protein antibody in the buffer. Time-resolved fluorescence (excitation, 320 nm; emission donor, 615 nm; emission acceptor, 665 nm) was monitored by using 2030 multilabel reader Victor5 (PerkinElmer). The readout was calculated as (acceptor counts/donor counts)×1000. The IC50 values were derived by fitting a sigmoidal dose-response curve to a plot of assay readout over inhibitor concentration. All fits were computed with the program Prism 5.03 (Graph Pad Software, San Diego, Calif.).

Exemplary compounds of the present invention were screened by the above mentioned assays and the results are tabulated; the Kd values (in range) of the selected compounds are set forth below in table-4 wherein “A” refers to a Kd value less than 0.05 μM, “B” refers to a Kd value in range of 0.05 μM to 0.5 μM and “C” refers to a Kd value greater than 0.5 μM.

TABLE 4
Kd values for CDK12 activity

| CDK12 Kd (μM) | Compound No. |
|---|---|
| A | 7, 8, 12, 14, 15 & 17-22 |
| B | 1, 6, 23, 24 & 26 |
| C | 9, 10, 13, 16 & 25 |

TABLE 5
Comparison data between CDK13 and CDK7

| Comp. | CDK13 | CDK7 |
|---|---|---|
| No | IC₅₀ μM | IC₅₀ μM |
| 1 | 0.024 | 11.4 |
| 3 | 0.018 | 1.90 |
| 4 | 0.004 | >10 |
| 5 | 0.024 | 1.70 |
| 6 | 0.5 | >10 |
| 7 | 0.005 | 1.021 |
| 8 | 0.0038 | 5.375 |
| 9 | 0.0169 | 5.45 |
| 10 | 0.804 | >10 |
| 11 | 0.096 | 2.118 |
| 12 | 0.0608 | >10 |
| 13 | 2.064 | >10 |
| 14 | 0.0039 | 1.117 |
| 15 | 0.2566 | 3.661 |
| 16 | 0.212 | >10 |
| 17 | 0.0536 | 1.556 |
| 18 | 0.119 | 5.453 |
| 19 | 0.013 | NA |
| 20 | 0.0362 | 2.868 |
| 21 | 0.0877 | 1.307 |
| 22 | 0.0209 | 5.304 |
| 23 | 0.28 | >10 |
| 24 | 0.236 | NA |
| 26 | 0.328 | NA |

*NA—Not available

We claim:

1. A compound of formula (I): 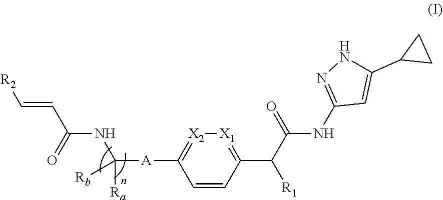
or a pharmaceutically acceptable salt thereof, an N-oxide or a stereoisomer thereof; wherein,
each of X₁ and X₂ is independently CH or N;
A is aryl, heteroaryl or a bond, wherein the aryl and heteroaryl are each optionally substituted with one or more substituents independently selected from halogen, alkyl and alkoxy;
R₁ is hydrogen or alkyl;
R₂ is hydrogen or —(CH₂)ₘ—NR_{c}R_{d};
Rₐ and R_{b} are each independently hydrogen or alkyl; alternatively, Rₐ and R_{b} together with the carbon atom to which they are attached form an optionally substituted cycloalkyl ring;
R_{c} and R_{d} are each independently hydrogen or alkyl; alternatively, R_{c} and R_{d} together with the nitrogen atom to which they are attached form an optionally substituted heterocyclic ring containing 0-2 additional heteroatoms independently selected from N, O and S;
m is 1, 2 or 3; and
n is 0, 1 or 2.

2. The compound of claim 1, having formula (IA): 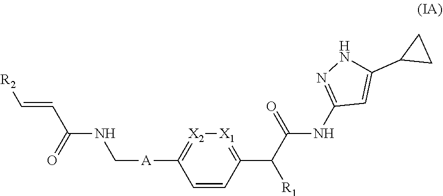
or a pharmaceutically acceptable salt thereof, an N-oxide or a stereoisomer thereof.

3. The compound of claim 1, having formula (IB): 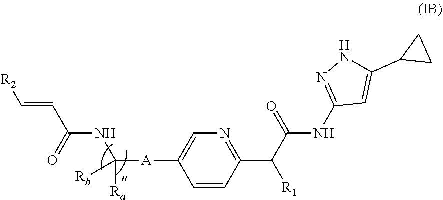
or a pharmaceutically acceptable salt thereof, an N-oxide or a stereoisomer thereof.

4. The compound of claim 1, having formula (IC): 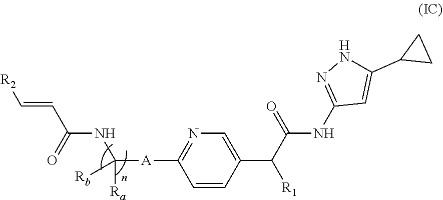
or a pharmaceutically acceptable salt thereof, an N-oxide or a stereoisomer thereof.

5. The compound of claim 1, having formula (ID): 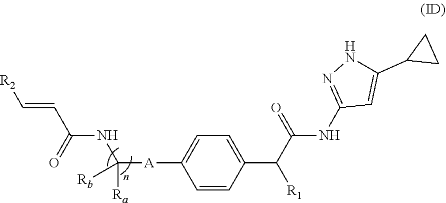
or a pharmaceutically acceptable salt thereof, an N-oxide or a stereoisomer thereof.

6. The compound of claim 1, having formula (IE): 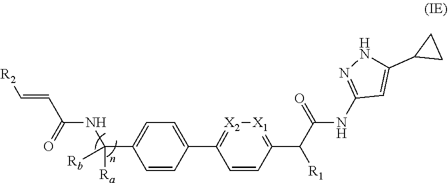
or a pharmaceutically acceptable salt thereof, an N-oxide or a stereoisomer thereof.

7. The compound of claim 1, having formula (IF): 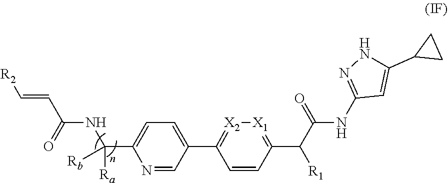
or a pharmaceutically acceptable salt thereof, an N-oxide or a stereoisomer thereof.

8. The compound of claim 1, having formula (IG): 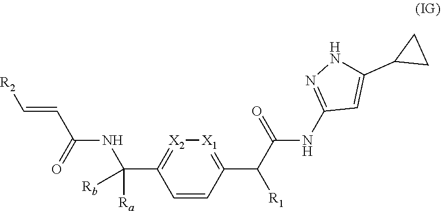
or a pharmaceutically acceptable salt thereof, an N-oxide or a stereoisomer thereof.

9. The compound of claim 1, having formula (IH): 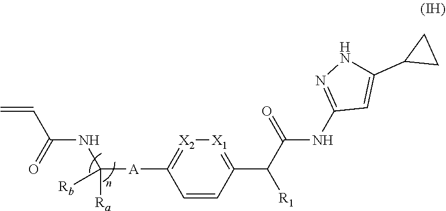
or a pharmaceutically acceptable salt thereof, an N-oxide or a stereoisomer thereof.

10. The compound of claim 1, having formula (IJ): 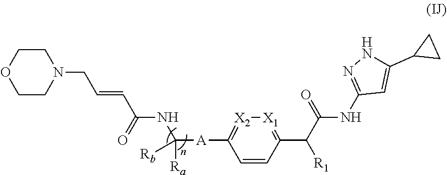
or a pharmaceutically acceptable salt thereof, an N-oxide or a stereoisomer thereof.

11. The compound of claim 1, having formula (IK): 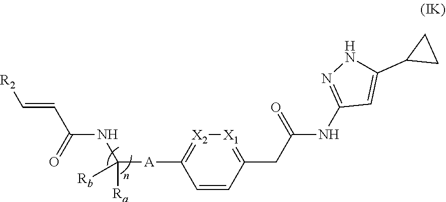
or a pharmaceutically acceptable salt thereof, an N-oxide or a stereoisomer thereof.

12. The compound of any of claims 1, 2 and 6-11, wherein, ring 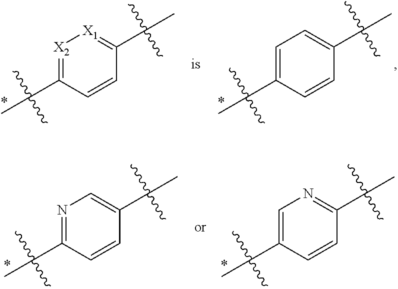
wherein * is the point of attachment with A.

13. The compound of any of claims 1-5 and 9-12, wherein, A is 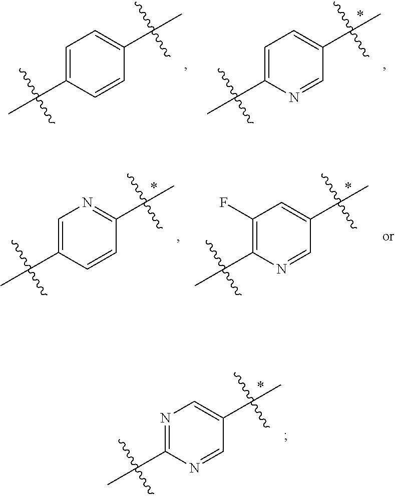 [SMILES: *c1ccc(*)cc1 ; *c1ccc(*)nc1 ; *c1ccc(*)nc1 ; *c1cnc(*)c(F)c1 ; *c1cnc(*)nc1]
wherein * is the point of attachment with ring

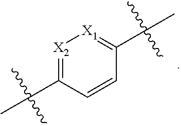

14. The compound of any of claims 1-8 and 12-13, wherein, R₁ is hydrogen or alkyl; R₂ is hydrogen or —(CH₂)ₘ—NR_{c}R_{d};
R_{c} and R_{d} together with the nitrogen atom to which they are attached form an optionally substituted heterocyclic ring containing 0-2 additional heteroatoms independently selected from N, O and S; and m is 1, 2 or 3.

15. The compound of any of claims 1-5 and 9-14, wherein, A is 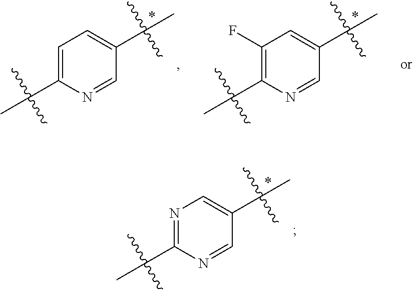 [SMILES: *c1ccc(*)nc1 ; *c1cnc(*)c(F)c1 ; *c1cnc(*)nc1]
wherein * is the point of attachment with ring 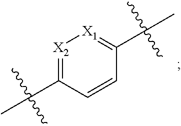
R₁ is alkyl, R₂ is hydrogen or —(CH₂)ₘ—NR_{c}R_{d};
R_{c} and R_{d} are each independently hydrogen or alkyl; wherein the alkyl is methyl; alternatively, R_{c} and R_{d} together with the nitrogen atom to which they are attached form an optionally substituted heterocyclic ring containing 0-2 additional heteroatoms independently selected from N, O and S; and m is 1, 2 or 3.

16. The compound of any of claims 1, 2 and 6-15, wherein ring 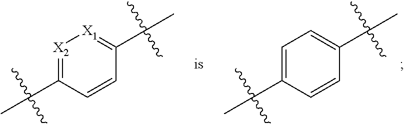
A is 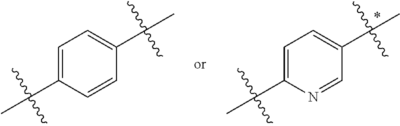 [SMILES: *c1ccc(*)cc1 ; *c1ccc(*)nc1]
wherein * is the point of attachment with ring 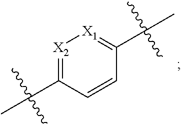
R₁ is alkyl;
R₂ is hydrogen;
Rₐ and R_{b} are each hydrogen; and n is 1.

17. The compound of any of claims 1-8 and 11-15, wherein R₂ is H or morpholinomethyl.

18. A compound selected from:

| Comp | |
|---|---|
| No. | NAME |
| 1 | N-(4′-(1-((5-cyclopropyl-1H-pyrazol-3- |
| | yl)amino)-1-oxopropan-2-yl)-[1,1′- |
| | biphenyl]-4-yl)acrylamide; |
| 2 | N-(4′-(1-((5-cyclopropyl-1H-pyrazol-3- |
| | yl)amino)-1-oxopropan-2-yl)-[1,1′- |
| | biphenyl]-4-yl)acrylamide (Isomer-1 |
| | of compound-1); |
| 3 | N-(4′-(1-((5-cyclopropyl-1H-pyrazol-3- |
| | yl)amino)-1-oxopropan-2-yl)-[1,1′- |
| | biphenyl]-4-yl)acrylamide (Isomer-2 |
| | of compound-1); |
| 4 | N-((5-(1-((5-cyclopropyl-1H-pyrazol-3- |
| | yl)amino)-1-oxopropan-2-yl)-[2,3 ′- |
| | bipyridin]-6′-yl)methyl)acrylamide; |
| 5 | (E)-N-(5-(4-(1-((5-cyclopropyl-1H-pyrazol- |
| | 3-yl)amino)-1-oxopropan-2- |
| | yl)phenyl)pyridin-2-yl)-4-morpholinobut- |
| | 2-enamide; |
| 6 | (E)-N-(5-(4-(1-((5-cyclopropyl-1H- |
| | pyrazol-3-yl)amino)-1-oxopropan-2- |
| | yl)phenyl)pyridin-2-yl)-4-morpholinobut- |
| | 2-enamide (Isomer-1 of compound-5); |
| 7 | (E)-N-(5-(4-(1-((5-cyclopropyl-1H-pyrazol- |
| | 3-yl)amino)-1-oxopropan-2- |
| | yl)phenyl)pyridin-2-yl)-4-morpholinobut- |
| | 2-enamide (Isomer-2 of compound-5); |
| 8 | (S)-N-((5-(4-(1-((5-cyclopropyl-1H- |
| | pyrazol-3-yl)amino)-1-oxopropan-2- |
| | yl)phenyl)pyridin-2-yl)methyl)acrylamide; |
| 9 | N-((4′-(1-((5-cyclopropyl-1H-pyrazol-3- |
| | yl)amino)-1-oxopropan-2-yl)-[1,1′- |
| | biphenyl]-4-yl)methyl)acrylamide; |
| 10 | N-((4′-(1-((5-cyclopropyl-1H-pyrazol- |
| | 3-yl)amino)-1-oxopropan-2-yl)-[1,1′- |
| | biphenyl]-4-yl)methyl)acrylamide (Isomer-1 |
| | of compound-9); |
| 11 | N-((4′-(1-((5-cyclopropyl-1H-pyrazol-3- |
| | yl)amino)-1-oxopropan-2-yl)-[1,1′- |
| | biphenyl]-4-yl)methyl)acrylamide (Isomer-2 |
| | of compound-9); |
| 12 | (E)-N-(4′-(1-((5-cyclopropyl-1H-pyrazol-3- |
| | yl)amino)-1-oxopropan-2-yl)-[1,1′- |
| | biphenyl]-4-yl)-4-morpholinobut-2-enamide; |
| 13A | (E)-N-((5-(4-(1-((5-cyclopropyl-1H-pyrazol- |
| | 3-yl)amino)-1-oxopropan-2- |
| | yl)phenyl)pyridin-2-yl)methyl)-4- |
| | morpholinobut-2-enamide; |
| 13 | (E)-N-((5-(4-(1-((5-cyclopropyl-1H- |
| | pyrazol-3-yl)amino)-1-oxopropan-2- |
| | yl)phenyl)pyridin-2-yl)methyl)-4- |
| | morpholinobut-2-enamide (Isomer-1 of |
| | compound-13A); |
| 14 | (E)-N-((5-(4-(1-((5-cyclopropyl-1H- |
| | pyrazol-3-yl)amino)-1-oxopropan-2- |
| | yl)phenyl)pyridin-2-yl)methyl)-4- |
| | morpholinobut-2-enamide (Isomer-2 of |
| | compound-13A); |
| 15 | (E)-N-((4′-(1-((5-cyclopropyl-1H-pyrazol- |
| | 3-yl)amino)-1-oxopropan-2-yl)-[1,1′- |
| | biphenyl]-4-yl)methyl)-4-morpholinobut-2-enamide; |
| 16 | (E)-N-((4′-(1-((5-cyclopropyl-1H-pyrazol- |
| | 3-yl)amino)-1-oxopropan-2-yl)-[1,1′- |
| | biphenyl]-4-yl)methyl)-4-morpholinobut- |
| | 2-enamide (Isomer-1 of compound-15); |
| 17 | (E)-N-((4′-(1-((5-cyclopropyl-1H-pyrazol- |
| | 3-yl)amino)-1-oxopropan-2-yl)-[1,1′- |
| | biphenyl]-4-yl)methyl)-4-morpholinobut- |
| | 2-enamide (Isomer-2 of compound-15); |
| 18 | (S)-N-(1-(5-(4-((5-cyclopropyl-1H- |
| | pyrazol-3-yl)amino)-1-oxopropan-2- |
| | yl)phenyl)pyridin-2-yl)cyclopropyl)acrylamide; |
| 19 | N-(1-(5-(4-((S)-((5-cyclopropyl-1H- |
| | pyrazol-3-yl)amino)-1-oxopropan-2- |
| | yl)phenyl)pyridin-2-yl)propyl)acrylamide; |
| 20 | N-(1-(5-(4-((S)-1-((5-cyclopropyl-1H- |
| | pyrazol-3-yl)amino)-1-oxopropan-2- |
| | yl)phenyl)pyridin-2-yl)ethyl)acrylamide; |
| 21 | (S)-N-((5-(4-(1-((5-cyclopropyl-1H- |
| | pyrazol-3-yl)amino)-1-oxopropan-2- |
| | yl)phenyl)-3-fluoropyridin-2-yl)methyl)acrylamide; |
| 22 | N-((6-(4-(1-((5-cyclopropyl-1H- |
| | pyrazol-3-yl)amino)-1-oxopropan-2- |
| | yl)phenyl)pyridin-3-yl)methyl)acrylamide; |
| 23 | N-(4-(1-((5-cyclopropyl-1H-pyrazol- |
| | 3-yl)amino)-1-oxopropan-2- |
| | yl)benzyl)acrylamide; |
| 24 | N-(4-(1-((5-cyclopropyl-1H-pyrazol- |
| | 3-yl)amino)-1-oxopropan-2- |
| | yl)benzyl)acrylamide (Isomer-1 of compound-23); |
| 25 | N-(4-(1-((5-cyclopropyl-1H-pyrazol- |
| | 3-yl)amino)-1-oxopropan-2- |
| | yl)benzyl)acrylamide (Isomer-2 of compound-23); |
| 26 | N-((5-(1-((5-cyclopropyl-1H-pyrazol-3- |
| | yl)amino)-1-oxopropan-2-yl)pyridin-2- |
| | yl)methyl)acrylamide; |
| 27 | N-((5-(4-(1-((5-cyclopropyl-1H-pyrazol- |
| | 3-yl)amino)-1-oxopropan-2- |
| | yl)phenyl)pyrimidin-2-yl)methyl)acrylamide; |
| 28 | N-((6′-(1-((5-cyclopropyl-1H-pyrazol-3- |
| | yl)amino)-1-oxopropan-2-yl)-[3,3′- |
| | bipyridin]-6-yl)methyl)acrylamide; and |
| 29 | N-((5-(2-((5-cyclopropyl-1H-pyrazol-3- |
| | yl)amino)-2-oxoethyl)-[2,3′-bipyridin]-6′- |
| | yl)methyl)acrylamide; |

or a pharmaceutically acceptable salt thereof, an N-oxide thereof or a stereoisomer thereof.

19. A pharmaceutical composition comprising a compound of any one of claims 1 to 18, or a pharmaceutically acceptable salt or a stereoisomer thereof and at least one pharmaceutically acceptable carrier or excipient.

20. The pharmaceutical composition of claim 19 for use in treating a subject suffering from a disease or condition associated with aberrant activity of CDK12/13.

21. The compound according to any one of claims 1 to 18, or a pharmaceutically acceptable salt or a stereoisomer thereof, for use as a medicament.

22. The compound according to any one of claims 1 to 18, for use in the treatment of a cancer, an inflammatory disorder, an auto-inflammatory disorder or an infectious disease.

23. The compound according to claim 21, for use in the treatment of a cancer.

24. The compound for use of claim 22, wherein the cancer is selected from the group consisting of a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma; hematopoietic tumors of lymphoid lineage, including leukemia, acute lymphoblastic leukemia, acute lymphocytic leukemia, Hodgkin's lymphoma, non-Hodgkin's lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

25. The compound of any one of claims 1 to 18, for use in the treatment of Myotonic Dystrophy type 1, Myotonic Dystrophy type 2, Fragile X associated tremor/ataxia syndrome, amylotrophic lateral sclerosis (ALS) and frontotemporal dementia, Huntington's Disease like 2, Huntington's Disease, several types of Spinocerebellar Ataxia, Dentatorubral-pallidoluysian atrophy and Spinal and Bulbar Muscular Atrophy.

26. A method of inhibiting CDK12/13 in a subject, comprising administering to the subject a compound of any one of the claims 1 to 18.

27. A method of treating or preventing diseases and/or disorders or condition mediated by CDK12/13 in a subject comprising administering to the subject in need thereof a therapeutically effective amount of a compound according to any one of the claims 1 to 18 or a pharmaceutically acceptable salt thereof.

28. The method of claims 26 to 27, wherein the CDK12/13 mediated disorder or disease or condition is selected from the group consisting of a cancer, an inflammatory disorder, an auto-inflammatory disorder and an infectious disease.

29. The method of claim 28, wherein the cancer is selected from the group consisting of a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma;
hematopoietic tumors of lymphoid lineage, including leukemia, acute lymphoblastic leukemia, acute lymphocytic leukemia, Hodgkin's lymphoma, non-Hodgkin's lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

30. The method of claim 27, wherein the disorder or condition mediated by CDK12/13 is Myotonic Dystrophy type 1, Myotonic Dystrophy type 2, Fragile X associated tremor/ataxia syndrome, amylotrophic lateral sclerosis (ALS) and frontotemporal dementia, Huntington's Disease like 2, Huntington's Disease, several types of Spinocerebellar Ataxia, Dentatorubral-pallidoluysian atrophy and Spinal and Bulbar Muscular Atrophy.

31. The method of any one of the claims 26 to 30, further comprising administering to the subject in need thereof one or more chemotherapeutic agents independently selected from anti-proliferative agents, anti-cancer agents, immunosuppressant agents and pain-relieving agents.

32. The method of any one of the claims 26 to 31, wherein the subject is a mammal including human.

33. Use of a compound of any one of claims 1 to 18 in the manufacture of a medicament for the treatment of cancer.
